(19) United States
(12) Patent Application Publication
(10) Pub. No.: US 2008/0041401 A1
(43) Pub. Date: Feb. 21, 2008

(54) Computer adjusted pressure wound care devices, systems & methods

(76) Inventors: Robert P. Casola, Naples, FL (US); Warren E. Parkhurst, North Fort Myers, FL (US)

Correspondence Address: DOONAN DWIGHT MCGRAW, 245 SAINT JAMES WAY, NAPLES, FL, 34104-6775, US

(21) Appl. No.: 11/893,273

(22) Filed: Aug. 15, 2007

Related U.S. Application Data
(60) Provisional application No. 60/837,724, filed on Aug. 15, 2006.

(51) Int. Cl. A61B 19/00

(52) U.S. Cl. 128/897

(57) ABSTRACT

A system and method of wound therapy treatment using differential subatmospheric pressures applied to the wound which are calculated to be at least the substantially optimum of such pressures after considering various wound conditions, and either continually or at intervals recalculating the best of such pressures to use. A control center contains all of the equipment for doing this, including a computer program. There are several different parts to the system which are important to the invention, some of which may also be considered to be individually patentable. This includes the computer program, the wound dressing features such as maintaining a seal around the wound by use of the differential subatmospheric pressure at the wound and also on the underside of the dressing that covers the wound, shaped foam or sponge-like wound fillers that are a part of the dressing, and are covered by the seal blanket of the dressing. These shaped devices fit the wound better than the current practice does, maintain their placement in the wound, and therefore do not permit tunnels or fistulas to develop inside the wound during the vacuum treatments of the wound.

Priority for this application is claimed based on the U.S. Provisional Patent Application Ser. No. US60/837,724, entitled: “Computer Adjusted Pressure Environment Vacuum Therapy Wound Care Devices, Systems and Methods” and filed on Aug. 15, 2006, by the same inventors who are the inventors named in this application. That application is hereby incorporated into this document by reference.

### BACKGROUND OF THE INVENTION

Prior art in the study of pressure-assisted healing appears to have started in Russia during the middle of the 20^{th} Century, although research in various forms of assisted healing was carried out in ancient times. Most recent and detailed research, as well as use, in the U.S.A. was done at the University of Maryland by several physicians, including Dr. Argenta, who later, at Wake Forest University in North Carolina, did more work in this field. His work there resulted in the patents by Messrs. Argenta et al, such as U.S. Pat. Nos. 5,645,081 and 5,636,643 granted in 1997, and patents including U.S. Pat. No. 7,216,651 that issued on May 15, 2007. That patent and the other two noted above trace their common history, at least in part, to the U.S. patent application Ser. No. 07/792,001, filed on Nov. 14, 1991. The References cited by the examiner that are listed as part of U.S. Pat. No. 7,216,651 include 118 U.S. Patents, and 18 Foreign Patents. There were also 343 “Other References” cited, which are presumed to have been by the applicants. Many of these citations bear no date in the citations listed in that patent. The applicants submitting this document have had no opportunity to review them for possible relevance. It is presumed that the examiner of that application did do so.

Most if not all of the commercially available devices and systems at the present time are based on the ideas and clinical trials detailed in those patents or the Russian studies with animals in the 1960s. Some of these and Russian studies relating to subatmospheric therapy are found in the “Other References” noted above. One of the problems with terminology relating to these devices and systems, which is reflected in the vast majority of case histories and further research projects carried out and reports made in the years starting in the 1960s since 1992, is that the basic theory is focused on something mistakenly called “Negative Pressure” which simply does not exist. Other terms used are “vacuum therapy” or words of an equivalent nature combined with the term “vacuum.” In the titles of many of the “Other References” cited in the U.S. Pat. No. 7,216,651 as noted below, terms such as “High-vacuum drainage,” “vacuum device,” “vacuum sealing,” “vacuum dressing,” “vacuum treatment,” “vacuum-assisted,” “vacuum-occlusion dressing,” “vacuum preparation of the wound,” “vacuum evaporative method,” “suction apparatus,” “suction device, “mini VAC device,” “mini vacuum drainage,” “closed-wound suction,” and the like are used at times. The two major terms are “negative pressure” with some variations, and “subatmospheric pressure,” or “subatmospheric pressure.” This appears to be a tacit recognition that there are pressures which are less than atmospheric pressure, but are still pressures that are positive, and that would apply not only on our own planet, but on others such as Mars, or even our moon, whose “ambient atmospheric pressures” are considerably less than that of Earth. For possibly that reason, it is now noted that the term “subatmospheric pressure” has become more prevalent starting primarily in the later 1990s, and certainly is more proper. Therefore, this term will usually be used hereafter.

Additionally, the articles in the historical literature seem not to recognize that it is the ambient atmospheric pressure existing at the time and place of the use of the devices and systems which imposes, because there is a differential subatmospheric pressure, the motive force acting from the ambient atmospheric pressure, which is a higher absolute pressure than the absolute pressure which is lower than the ambient atmospheric pressure by the amount of the differential subatmospheric pressure, to assist in the healing process. Atmospheric pressure is variable, not constant, and varies with temperature and the altitude above sea level as well as the presence of various weather factors, such as relatively localized high pressure systems and low pressure systems which may be present at any particular time. Thus, the atmospheric pressure even at the same altitude can vary with time, and therefore it is the currently existing ambient atmospheric pressure, which is the net atmospheric pressure considering all of these variables, that is important. Unless pressure-assisted healing is based upon the current differential pressure existing between the current ambient atmospheric pressure and the lower-than-atmospheric (subatmospheric) pressure currently being applied to the wound, and not on some standard negative pressure which is applied irrespective of the ambient atmospheric pressure, the desired wound-healing results are not always attained. Without going into much detail, there can be a differential subatmospheric pressure of 10 mm Hg on one day, when the ambient atmospheric pressure was 970 mm Hg, so that the ambient atmospheric in an absolute lesser pressure of 960 mm Hg, and on the next day, when the ambient atmospheric pressure at the same place is 950 mm Hg. On that day, if the differential subatmospheric pressure of 10 mm Hg is still to be used, the absolute lesser pressure would be 940 mm Hg. Therefore, the lower absolute pressure on the first day is higher than the ambient atmospheric pressure the next day. Such is the case with the currently employed devices. While even though one or two of the currently employed devices have a manual-input-control for setting the “negative pressure,” there were, and still are, no definitive guidelines issued that have been tested on human beings and are in place to accurately identify what value of that “negative pressure” should be employed for a particular wound suffered by a particular patient, not only at the time of beginning treatment, but throughout the treatment using a subatmospheric pressure. It seems that it has been, at best, just a guessing game when it is not even considered that the system should be normally employed to set any different differential atmospheric pressures from the one set initially that would be preferred because of changes, particularly within the wound, that are being sensed, much less setting it automatically yet subject to revision by the person tending the treatment session.

In recent years, wounds which are open have often been treated by devices using what has been termed “Negative Pressure” applied to a wound in the manner which is the subject of several U.S. Patents assigned to Kinetic Concepts, Inc., of San Antonio, Tex., U.S.A., or one of its more recent corporate entities (herein after referred to as “KCI™”), as well as their having taking licenses or purchased patents from others. Similar treatment devices and systems are also the subject of several U.S. Patents assigned to a company named Blue Sky™. After the filing of the provisional patent application on which this application is based, the firm of Smith and Nephew has purchased Blue Sky. Other companies, including Hill-Ron™, located in Indiana, and Medela™, headquartered in Switzerland, have patents in the area of immediate interest.

KCI has promulgated an “Articles Book” in which there are discussions of the use of the vacuum technology espoused by that company. KCI™ has also promulgated an “Abstracts Book” which is a collection of case reviews where its devices and systems were used. Unfortunately, although there is considerable information about the physiology and chemistry of wound treatment on a great many types of wounds, these publications do not provide a basis for the understanding of the basic physics involved, and do not seem to have even recognized the true basic physics involved. The devices and systems used by KCI™ for some years have used a set “negative pressure” of 125 mm Hg. Such a pressure is so numerically high that its use in that manner has often been proven detrimental to healing. Some time later, KCI™ began to use a cyclic off-on-off-on system, still supplying that same 125 mm Hg “negative pressure,” apparently trying to “average out” the applied “negative pressure” as later discussed below and illustrated in one of the drawing Figures. This procedure is claimed in at least one of their patents. They also for years have been using in the wound a sponge-like foam implant which has parallel sides forming a square in cross-section or a rectangle that is nearly a square in cross-section, and which has simply been cut to the length of the wound. Recently, KCI™ has offered a series of foam implants which are of different sizes but still have the same style of cross-section. That is still not providing a more precise fitting of the implant to the actual shape of the wound. This “one-type-fits-all” approach has functioned satisfactorily at times, but not every time. In particular, it does not function properly when the wound sides are not substantially parallel (and they are seldom if ever parallel) while their foam implant has parallel sides. There have been several reports of problems in healing occurring when the 125 mm Hg and the parallel-sided inserts are used. All too often their use has actually resulted in the formation of at least partially sealed tubular portions or tunnels, sometimes referred to as fistulas, within the wound that become infected, and require weeks of antibiotic and other types of treatment to overcome the infection and allow the wound to then heal properly, without further use of the KCI™ device and system earlier employed on that wound. Cases of this malfunction have been fully documented. Blue Sky™, however, only useD gauze layers, and a recent patent infringement case tried by a federal District Court in Texas before a jury, held that Blue Sky™ did not infringe the claims of any patents that KCI™ and/or Wake Forest University™ charged Blue Sky with infringement. Since that decision is being appealed by both parties, including Smith and Nephew, who purchased the Blue Sky company, it is far from being settled at the time of the filing of this regular patent application.

### FIELD OF THE INVENTION

The invention relates to improvements in the differential subatmospheric pressure treatment, often referred to by others as vacuum therapy treatment, of various types of wounds and the devices, systems and methods for practicing such improvements. More particularly, the invention relates to the amount of subatmospheric differential pressure or, in a term sometimes used, the amount of vacuum wound therapy applied to the wound. The more important factors that are considered in setting and then modifying the applied subatmospheric differential pressure as needed for a particular wound include the subatmospheric differential pressure actually in the wound, the size of the wound (which changes as proper healing progresses), the temperature inside the wound, and the patient's sensitivity to the procedure. There are also other factors that are related to the amount of subatmospheric differential pressure being maintained in the wound which can relate to the applied subatmospheric differential pressure. Usually, the patient-sensitivity factor is determined and remains constant, and is manually entered before the treatment session begins, but even it can change to being more sensitive or less sensitive. When it does, the new sensitivity is entered and has an immediate effect on the applied subatmospheric differential pressure. The area or volume of the wound changes, but usually not over a short period of time. Therefore, it is also entered before the treatment session begins, but can be changed when it changes. There are now some devices that will at set intervals determine the area or the volume of the wound. If such a device is used, then its data can also be used for automatically determining whether or not any change in the applied subatmospheric differential pressure is needed because of such change. The more changeable factors that are being sensed result in a measurement of the information of each factor's being sensed as being data which are entered into a control center. The control center has, among other things, a computer unit and a computer program, which is also a part of the invention. It also includes a device that can receive a recommended change in the applied subatmospheric differential pressure, considering all of the factors that are being sensed and their measurements of the sensed factors. The computer unit receives the data being sent, and already has the data that was entered before the treatment session started. That data may be displayed. Then, either continuously and virtually instantaneously, or at desired short time intervals on the order of seconds or a very few minutes, the computer unit, using the proprietary computer program, recomputes the preferred differential subatmospheric pressure for the conditions sensed. When these recomputations indicate that any of the data changes sufficiently to modify the current differential subatmospheric pressure being applied to the wound, the recommended change may also be displayed. The computer unit also sends that recommended change to the control for setting the applied subatmospheric differential pressure, which then causes that change to be implemented. This is preferably an adjustment to the speed of the air impeller of the pump. When more or less subatmospheric differential pressure should be applied, this adjustment to the pump will accomplish that change. The differential subatmospheric pressure being applied to the wound is then at or very close to optimum conditions for the wound's healing. Because that modified applied subatmospheric differential pressure is displayed, the person in charge of the treatment session, who is authorized to do so, may either cancel or modify the change, when that is considered to be appropriate. The invention includes the system as a whole, with the system having various types of parts, a few of which are mandatory. It includes the concept of continuously monitoring certain conditions and having the computer unit cause the change to the applied differential subatmospheric pressure being supplied to the wound. This is preferably accomplished by the computer program as it receives the data from various sensors for determining the desired treatment, and sending data relating to those determinations of those certain appropriate conditions to the control mechanism which controls the output of the subatmospheric pressure, and adjusting that applied subatmospheric pressure as needed to maintain the desired therapy treatment with all of the conditions that exist. The program used is capable of defining the the type and characteristics of the particular wound dressing to be used on the patient in view of all of the sensed conditions. The invention also includes the methods of doing these steps that result in a change to the applied subatmospheric pressure being applied to the wound.

### DESCRIPTION OF THE RELATED ART

In addition to that related art noted in the background of the invention, the following U.S. patents are also of interest in relation to the invention:

U.S. Pat. No. 4,969,880, entitled: “WOUND DRESSING AND TREATMENT METHOD,” issued: Nov. 13, 1990; Inventor: Zamierowski; Assignee: None shown.

U.S. Pat. No. 5,100,396, entitled: “FLUIDIC CONNECTION SYSTEM AND METHOD,” issued: Mar. 31, 1992; Inventor: Zamierowski; Assignee: None noted. The portion of the term of this patent subsequent to Nov. 13, 2007, has been disclaimed. It is a Continuation-In-Part (CIP) of U.S. Pat. No. 4,969,880, noted above.

U.S. Pat. No. 5,261,893, entitled: “FASTENING SYSTEM AND METHOD,” issued: Nov. 16, 1993; Inventor: Zamierowski; Assignee: None noted. The portion of the term of this patent subsequent to Nov. 13, 2007, has been disclaimed. It is a CIP of U.S. Pat. No. 5,100,396, which is a CIP of U.S. Pat. No. 4,969,880, noted above.

U.S. Pat. No. 5,527,293, entitled: “FASTENING SYSTEM AND METHOD,” issued: Jun. 18, 1996; Inventor: Zamierowski; Assignee: Kinetic Concepts, Inc. It is a CIP of U.S. Pat. No. 5,261,893, which is a CIP of U.S. Pat. No. 5,100,396, which is a CIP of U.S. Pat. No. 4,969,880. There is no disclaimer shown of any part of the patent.

U.S. Pat. No. 5,636,643, entitled: “WOUND TREATMENT EMPLOYING REDUCED PRESSURE,” issued: Jun. 10, 1997; Inventor: Argenta et al; Assignee: Wake Forest University; and U.S. Pat. No. 5,645,081, entitled: “METHOD OF TREATING TISSUE DAMAGE AND APPARATUS FOR SAME,” issued Jul. 8, 1997; Inventor: Argenta et al; Assignee: Wake Forest University, both of which claim, as a whole, or at least in part, the same application as a parent of their claimed invention.

U.S. Pat. No. 7,216,651, entitled “WOUND TREATMENT EMPLOYING REDUCED PRESSURE,” issued May 15, 2007; Inventor: Argenta et al; Assignee Wake Forest University, and also claiming the same application as a parent, at least in part, as do the above two patents that also list Argenta et al as the inventors, and Wake Forest University the assignee.

U.S. Pat. No. 6,071,267, entitled: “MEDICAL PATIENT FLUID MANAGEMENT INTERFACE SYSTEM AND METHOD,” issued Jun. 6, 2000; Inventor: Zamierowski; Assignee: Kinetic Concepts, Inc.

U.S. Pat. No. 6,142,982, entitled: “PORTABLE WOUND TREATMENT APPARATUS,” issued: Nov. 7, 2000; Inventor: Hunt, et al; Assignee: KCI Medical Limited

U.S. Pat. No. 6,203,563, entitled: “HEALING DEVICE APPLIED TO PERSISTENT WOUNDS, FISTULAS, PANCREATITIS, VARICOSE ULCERS, AND OTHER MEDICAL OR VETERINARY PATHOLOGIES OF A PATIENT,” issued Mar. 20, 2001; Inventor: Fernandez; Assignee: (none noted.)

U.S. Pat. No. 6,174,306, entitled: “DEVICE FOR VACUUM-SEALING AN INJURY,” issued Jan. 16, 2001; Inventor: Fleischmann; Assignee: (none noted.)

U.S. Pat. No. 6,458,109, entitled, “WOUND TREATMENT APPARATUS,” issued Oct. 1, 2002; Inventor: Henley et al; Assignee: Hill-Rom Services.

U.S. Pat. No. 6,500,112, entitled, “VACUUM DOME WITH SUPPORTING RIM AND RIM CUSHION,” issued: December, 2002; Inventor: Khouri; Assignee: Brava, LLC

U.S. Pat. No. 6,695,823, entitled, WOUND THERAPY DEVICE,” issued Feb. 24, 2004; Inventor: Lina et al; Assignee: KCI Licensing, Inc. Applicant claimed priority under 35 U.S.C., Section 119 of U.S. code, for Provisional Application Ser. No. 60/128,567 filed Apr. 9, 1999.

U.S. Pat. No. 6,752,794, entitled, “VACUUM THERAPY AND CLEANSING DRESSING FOR WOUNDS,” issued Jun. 22, 2004; Inventor: Lockwood, et. al; Assignee: Hill-Rom Services, Inc.

U.S. Pat. No. 6,755,807, entitled, “WOUND TREATMENT APPARATUS,” issued Jun. 29, 2004; Inventor: Risk, Jr. et al; Assignee: Hill-Rom Services, Inc.

U.S. Pat. No. 6,764,462, entitled, “WOUND TREATMENT APPARATUS,” issued Jul. 20, 2004; Inventor: Risk, Jr. et al; Assignee: Hill-Rom Services, Inc.

U.S. Pat. No. 6,767,334, entitled, “WOUND TREATMENT APPARATUS,” issued Jul. 27, 2004; Inventor: Randolph; Assignee: KCI Licensing, Inc.

U.S. Pat. No. 6,800,074, entitled, “WOUND TREATMENT APPARATUS,” issued Oct. 5, 2004; Inventor: Henley et al; Assignee: Hill-Rom Services, Inc.

U.S. Pat. No. 6,824,533, entitled, “WOUND TREATMENT APPARATUS,” issued Nov. 30, 2004; Inventor: Risk, Jr. et. al; Assignee: Hill-Rom Services, Inc.

U.S. Pat. No. 6,887,228, entitled: “TREATMENT OF WOUND OR JOINT FOR RELIEF OF PAIN AND PROMOTION OF HEALING,” issued: May 3, 2005; Inventor: McKay; Assignee: (none noted.)

U.S. Pat. No. 6,936,037, entitled, “TISSUE CLOSURE TREATMENT SYSTEM, PATIENT INTERFACE AND METHOD,” issued Aug. 30, 2005; Inventors: Bubb and Zamierowski; Assignee: KCI Licensing, Inc.

U.S. Pat. No. 6,951,553, entitled, “TISSUE CLOSURE TREATMENT SYSTEM AND METHOD WITH EXTERNALLY-APPLIED PATIENT INTERFACE,” filed Dec. 31, 2002; issued Oct. 4, 2005; Inventors: Bubb and Zamierowski; Assignee: KCI Licensing, Inc.

U.S. Pat. No. 6,979,324 entitled, “CLOSED WOUND DRAINAGE SYSTEM,” issued Dec. 27, 2005; Inventors Bubb and Zamierowskil; Assignee: KCI Licensing, Inc.

Referenced Medical Article: National V.A.C. 2004 Educational Conference, “ARTICLES”, Journal of Plastic and Reconstructive Surgery. “Closing Time” By Dr. Thomas Sunog: “ . . . Due to the vac's ability to pull interstitial fluids from the wound, it wasn't long before two separate tunnels were discovered in the wound. Had the wound closed with the tunnels intact, a much more serious situation would have developed.” It so happens that one of the inventors hereto has a well-documented case (discussed later) in which that closure of the tunnels occurred, and it became much more serious situation.

There may be some relevance found in one or more of the 343 “Other References,” the 18 foreign patents, and the 118 U.S. Patents cited in the U.S. Pat. No. 7,216,651, noted above, but, at the time of filing, this is very simply beyond the scope of the inventors or their assignee.

### BRIEF SUMMARY OF THE INVENTION

The most simple, and therefore the broadest, form of the invention is a system and a process being carried out, wherein a wound being treated uses a differential subatmospheric pressure created by a subatmospheric pressure source and is being controllably delivered to the wound, and there are one or more sensors that sense different conditions relating to the wound and send data representing each of those sensed different conditions is sent to a computer that has the data that defined the differential atmospheric pressure then being applied to the wound. That computer receives and stores all received data and is constantly calculating the effects, when any one or more of those changing conditions, which can benefit from a change in the current differential subatmospheric pressure calculated by the computer, changes from a previously sensed condition differential subatmospheric pressure value that is currently being sent to the wound, to the extent that the computer takes into account all of the sensed conditions, as well as conditions of the particular patient that have been entered into the computer as values, calculates the net changes that should be made in the current subatmospheric pressure created and delivered to the wound, and generates a control signal requiring any such net change to be made in the current subatmospheric pressure be executed, causing the pressure source to modify the differential subatmospheric pressure being delivered to the wound accordingly.

The most-preferred system is comprised of the following items, and systems with less than all of these items but still functioning to be a computer information-adjusted pressure environment system and using the method or process aspects of the invention. The omission of some of these items from a system being used, resulting in a simpler system, can still be within the purview of the invention, and are also preferred:

A master controller device which contains or is connected with the following items, (a few of which may be omitted if cost is a major factor, and at times some manual operations may be substituted) are acceptable and still within the purview of the invention.:
A pump with which to establish a subatmospheric differential pressure from within the wound to a point where atmospheric pressure exists.
A control mechanism which utilizes a sensing device to sense and measure the temperature at the wound site at any time. Such a device could be a Thermistor, which is typical of one of several equivalent temperature-sensing and measuring devices.
A control mechanism which utilizes data from a sensing device, such as but not limited to a piezoelectric device which is only an example, which measures the magnitude of the imposed differential pressure between the wound and the ambient atmospheric pressure at any time and at any place where the patient is located. There are also other types of sensors that are known to sense pressures.
A control mechanism which utilizes data from a sensitive flow meter which measures the flow of the interstitial fluids as they are being removed from the wound.
A control mechanism which utilizes data from a Thermister or equivalent already-known temperature-measuring-and-signal-generating device which measures the temperature of the interstitial fluids as they are being removed from the wound.
A control mechanism with which to control the fluctuation of the imposed differential pressure within narrow process-control design limits.
Electronic circuits and devices to acquire at least the pressure and temperature data, and if there are other data relating to the condition of the wound such as the flow rate and temperature of the interstitial fluids as they are removed from the wound because of a differential subatmospheric pressure in the wound, and to record all such sent and received data on removable magnetic or other recording media such as DVD or CD or CD-Rom discs, hard drives or flash drives, or any similar devices that are, or become, currently available.
Electronic circuits and devices to record the values of wound dimensions and the selected treatment mode input into the control mechanism, and to convert these to computer files for later readout and examination as well as using them in its calculations toward obtaining a substantially optimum differential subatmospheric pressure to be applied to the wound, and, if desired to have the capability of adjusting the supply source of the subatmospheric pressure to produce that substantially optimum differential subatmospheric pressure subject to it's being overridden by a medical attendant when that is considered to be needed for the good of the patient.
Rechargeable batteries and connections to a main electrical supply in order to supply the necessary electric power to the devices detailed above. The system and the devices are to be able to operate either on main electrical power or on batteries for portability.
Means to attach and detach some or all of the devices of the inventive system to be claimed, and particularly at least major parts of the control center and the backup battery and optionally the container for holding the removed interstitial fluids, to or from whatever stationary or moveable object is to be used in the treatment of the wound, or on a support device for supporting the elements of the system during use of the system and, optionally, being used as well as when the system has to be transported or stored.
Means to stop operations and/or sound an alarm if preset limits on certain conditions are not met or exceeded.
Means to facilitate the storage and disposal of the interstitial fluids removed from the wound being treated.
A positive differential pressure sealing blanket to be placed over the wound to contain the differential pressure to be imposed and which contains means to effect a seal between atmospheric pressure and the patient being treated and the electronic sensors to measure pressures and temperatures in real time.
The sealing blanket that is sealed to the patient's body around the wound by the force of the ambient atmospheric pressure acting on its outer surface while the lower pressure, which is lower than that atmospheric pressure by the amount of differential atmospheric pressure being applied to the wound, is acting on the inner surface of the sealing blanket.
A connecting apparatus between the controller device and the sealing blanket to allow collection of expelled interstitial fluids from the wound walls during the healing process (generally a simple plastic tube) and the means necessary to transmit the data on pressure and temperature, and possibly other data being collected from the wound area, to the control mechanism. This apparatus preferably contains quick connect/disconnect means of attaching/detaching it to/from the controller device and the Sealing Blanket.
Wound dressing materials of the proper shape, tensile strength, pore size distribution, porosity percent, compressibility, structural integrity, permeability to fluids, chemical reactivity, and retention of these properties to the extent necessary when exposed to the differential pressure to be imposed. The wound dressings to be properly shaped to resist collapse under all conditions to be imposed, and placed in the wound in such a way as to embrace the wound walls at all values of differential pressure to be imposed and at substantially all times.
Wound dressings made in substantial accordance with the wound dressings illustrated in FIGS. 5, 6, 7 8, and 9, and possessing the pore size distribution characteristics shown in FIG. 11 and the compressibility characteristics for Sample 1 shown in FIGS. 12 and 13.

### BRIEF DESCRIPTION OF THE DRAWINGS

FIG. 1 is a schematic representation of the system embodying the invention.

FIGS. 2, 3, and 4 show cross-sectional views of a wound undergoing differential subatmospheric pressure-assisted healing. FIG. 2 shows a wound fitted with a standard production wound dressing of the rectangular shape that has been used in the vast majority of wound care equipment for some years. FIG. 3 shows the developing condition as the treatment continued for about a week, and FIG. 4 shows the condition developed after continuing the same treatment for another period of time.

FIGS. 5 and 6 show the differential pressure blanket components which seal the differential subatmospheric pressure and any healing fluids produced within the wound cavity and the wound dressing allowing collection thereof, and which contains the measuring devices that supply continuous data to measure the progress of healing and to control the differential subatmospheric pressures. The insert in the dressing has been shaped in cross-section to fit the sides of the wound to be treated. FIG. 5 shows the dressing and the insert in place before the system is turned on. FIG. 6 shows the dressing, insert, and blanket after the process has started, during which time the elements that are compressible are compressed, the blanket having been compressed to about half of its unpressed thickness

FIGS. 7, 8, and 9 show cross-sectional views of a wound undergoing differential pressure-assisted healing. FIG. 7 shows a wound fitted with an inserted wound dressing having appropriate strength, porosity, pore size distribution, permeability and structural integrity in one of the selected modes of operation of the system. The inserted dressing also has a shape, shown in cross-section, that fits the sides of the wound. As the wound is relatively large, the computer program in the control mechanism has set the differential subatmospheric pressure to a lesser amount, about 50 mm Hg, before the start of treatment, day 0, and the treatment is then started and that first day of treatment is day 1, let's say about noon. FIG. 8 illustrates the condition of the wound at about noon of the 7^{th} day of treatment. The wound has healed nicely, and is smaller in volume and area, so the computer program, operating constantly and sensing the changes in wound size and other sensed parameters, including the pressure actually in the wound, that could have an effect on the amount of differential subatmospheric pressure to be continually used. As a result, it has increased the amount of differential subatmospheric pressure in increments during this first week of treatment to about 90 mm Hg. It should be noted that this setting will generate the about the same force tending to close the wound as at the beginning of day 1. About a week later, FIG. 9 begins with the 14^{th} day of treatment, also at about noon. The control mechanism is now allowing a control setting of the differential subatmospheric pressure to be increased to about 125 mm Hg, which will generate a force tending to close the wound that has the same value as in FIGS. 7 and 8, due to the ever smaller area of the walls of the wound as healing progresses. As the wound size decreases and the insert becomes smaller with the increased pressure, a greater differential subatmospheric pressure will result in about the same force being generated because it is now acting on a much smaller area.

FIG. 10 shows the calculation of the magnitude and direction of forces imposed on the wound walls of a sample wound by a differential subatmospheric pressure of 75 mm Hg.

FIG. 11 has a graph which shows, as an example, the tests which must be run to select the optimum wound dressing material considering the property of Pore Size Distribution.

FIG. 12 is a graphic presentation of the example of the trend line developed for the successful insert candidates' materials passing the pore size distribution tests of FIG. 11.

FIG. 13 shows the results of a simple test of the sample wound dressing mentioned above, identified as Sample 1 concerning FIGS. 10 and 11, using a commercial sample known in the trade as GranuFoam™, which is in widespread use today, after having undergone a simple alternating compression-and-relaxation test cycle for a period of 24 hours. In this specific test, a load of approximately 45 PSI applied for approximately 200 compression-relaxation cycles to two different samples. As can be seen, the comparison in the photo of a piece of the Sample No. 1 not tested vs. the Sample No. 1 after testing shows that the material would not be suitable for use as a wound dressing, as, due to its structural collapse, it would not have remained in contact with the wound walls for the period of time from installation to the time of the change of the dressing. Conversely, the other proposed wound dressing, having different characteristics and made of a different material, identified as Sample 2, showed no sign of structural collapse after having undergone the same test, and it regained its original shape immediately (within a few seconds) after removal of the forces of the test. Sample 2 was subjected to a further test of the same compression—relaxation regime for one month. Some slight deformation was then observed, but when the sample was washed and dried, it immediately resumed its original behavior, making it a strong candidate as a wound dressing material when this property is considered.

FIG. 14 shows a sample of GranuFoam™ obtained from KCI™, having a length of 3.5 inches, a width of 2.5 inches, and a thickness of 1.375 inches. It was subjected to a force imposed on its end (which had an area of 3.44 square inches), acting to decrease its length, ranging from 0 to 7.0 pounds which was from 0 psi to almost 2.35 psi. It was compressed continuously until it measured only 0.4 inches. At this point, the foam, which is much like a sponge, had compressed to the point that it was nearly solid, and would have possessed porosity and permeability only in minute quantities. It demonstrates the basic physics involved in foam collapse during service application and operation in wound VAC therapy.

FIG. 15 is a graphic depiction of the results of the construction of a laboratory device with which to measure the compressibility of a sample of wound dressing material and its application to the test of a type of wound dressing presently in wide usage.

FIG. 16 shows the results of compression tests carried out on three different thicknesses of otherwise similar samples of the GranuFoam™ used by the major supplier of VAC systems and the accompanying supplies. These samples were purchased from that supplier. All samples were the same size in length and width, but had three different thicknesses.

FIG. 17 is a three-dimensional representation of a wound as it was pressure-compressed. The sample used was made of GranuFoam™ having a standard rectangular cross-section. Such an insert is shown in FIGS. 6 and 7 after it has been initially installed but before it had been subjected to any pressure.

FIG. 18 is a graph showing the actual subatmospheric pressure in a particular volume simulating the volume of a typical wound, wherein the target pressure is 650 mm Hg, but is maintained within a range of 635 mm Hg to 665 mm Hg by turning a vacuum pump on and off. This is what the current major producer of the VAC system has done for some time in order to maintain a subatmospheric pressure in a wound which is to be kept nominally at 650 mm Hg

FIG. 19 shows an example of the changeable nature of the differential subatmospheric pressure within the wound due to the pressure control settings of the control mechanism of the invention herein disclosed and claimed. The figures given for absolute pressure settings are illustrative only.

FIG. 20 shows the progression of healing in an actual case studied in 2005. The data on wound volume (length×width×depth) and the surface area of the wound (length×width) are plotted against the time of healing.

FIG. 21 is a graph showing the actual progress, regression, and eventual healing of a wound that had developed infected tunnels as shown in FIGS. 3 and 4, and the bad results that followed for that patient.

### HISTORY OF THE RELATED ART

In order to more fully understand and appreciate the invention or inventions herein disclosed in its various aspects, including devices, systems, and methods, additional background information should become familiar and understandable to anyone who desires to make, use or sell devices incorporating any of the devices, systems, and methods of the invention or inventions. The present commercially used art, and the prior art found in various patents and literature, and particularly that which has been used and sold by one or more organizations in recent years, and through the period leading up to the filing of this patent application, should be presented and explained.

Prior art in the study of pressure-assisted healing appears to have started in Russia during the middle of the 20^{th} Century, although research in various forms of assisted healing was carried out in ancient times. Most recent and detailed research in the USA was started in 1992 and resulted in the patents of Messrs Argenta et al. (Researchers at Wake Forest University in North Carolina) U.S. Pat. Nos. 5,645,081 and 5,636,643 granted in 1997. Most commercially available devices and systems at the present time are based on the ideas and clinical trials detailed in those patents or the Russian studies.

One of the more serious problems encountered by the suppliers of the devices and those who attach the devices to a patient is one that is reflected in the vast majority of case histories and further research projects carried out in the years since 1992. While at first it appears to be simply a question of semantics, it in fact confuses those that are involved in making, selling and using “Negative Pressure Therapy for the healing of wounds” and think that negative pressure as the words clearly state actually exists. They sometimes make decisions based on that perception which are not correct decisions for the best interests of some patients. That problem is that the basic theory is focused on something called “Negative Pressure” which simply does not exist. In order for it to be defined, one has to establish the definition of the absolute dividing line between “positive pressure” and “negative pressure.” If such a dividing line were to exist, it would have to be the pressure that we would call “absolute zero pressure.” By comparison to that which is more commonly known, it would be the pressure equivalent of temperature which is known as “absolute zero temperature.” All temperatures that exist are always positive relative to that absolute zero value. By definition, there simply cannot be a “negative temperature” which is lower than the absolute zero temperature.

Following that analogy, then we would be required to posit the existence of a physical state or condition in which there is less than absolute zero pressure, but, by definition, there cannot be any lower temperature than absolute zero temperature. Similarly, also by definition, there cannot be any lower pressure than absolute zero pressure. Therefore, factually, there is no such thing as “negative pressure.” This has led to many misunderstandings by the medical practitioners making use of the devices available at this time, and, unfortunately, to some unnecessarily adverse medical consequences. Additionally, many of the articles in the historical literature seem not to recognize that it is the ambient atmospheric pressure which imposes moving forces on the air, in this case as well as on the wound tissue, through a differential pressure, which is the pressure difference between the ambient atmospheric pressure and a pressure that is lower than the ambient atmospheric pressure. Too often, it apparently seems to be more understandable to the writers to say that the “vacuum pressure” moves something. In other areas of endeavor the common reference to that subatmospheric pressure just uses terms such as partial vacuum, or just vacuum. A “vacuum pressure” is just a pressure that is lower than the ambient air pressure at the time and place where it is physically located. Yet, it is still greater than absolute zero pressure. This differential subatmospheric pressure is simply the value portion of the ambient atmospheric pressure that exerts the real motive force that moves air through the wound at a pressure lower than ambient absolute atmospheric pressure and thus assists in the healing process.

If one goes back to first principles, it is the earth's gravity which is the motive force behind pressure-assisted healing. This natural gravitational force has, over billions of years, attracted and held a blanket of gasses (primarily nitrogen and oxygen) around the surface of our planet Earth that we call “air” or “the atmosphere.” It is this gravitational force acting upon this blanket of gasses which creates what we call atmospheric pressure. This pressure is variable, not constant, and varies with temperature and the altitude above or below sea level It also varies with changes in weather conditions where there are what aerologists term high-pressure and low-pressure areas which move generally eastward over the earth's surface. One often sees dramatic illustrations of these pressure area movements on television weather broadcasts, and they are often mentioned in radio weather broadcasts, particularly when hurricanes or typhoons are being reported. Those who experienced hurricanes or typhoons in 2005 and 2006 that devastated parts of the U.S. southern coast and parts of the China-Japanese area are very much aware of the strength that even those subatmospheric differential pressures carry with them. They are often surprised that a 125 mm Hg decrease in pressure is equal to about 2.4 psi, and that is the pressure differential in a Category 4 hurricane. That puts the “negative pressure” of 125 mm Hg that has been suggested by suppliers of the vacuum wound therapy in the same category insofar as a decrease in absolute ambient air pressure is concerned, in the same category as a Category 4 hurricane.

Pressure-assisted healing properly should be based upon a “differential pressure,” referenced always to the currently existing ambient atmospheric pressure at the location that the treatment is taking place from which the differential is applied. The differential pressure can be either super-atmospheric or subatmospheric. In vacuum treatment of wounds, the differential pressure used is always a subatmospheric differential pressure. In such wound treatment, the subatmospheric differential pressure being exerted in the wound should be able to have the settings automatically changed in accordance with changes in the existing ambient atmospheric pressure so that pressure-assisted healing can have the desirable subatmospheric differential pressure is always being applied, even though the ambient atmospheric pressure sometimes drastically changes because of weather systems. There is common recognition that the ambient atmospheric pressures that normally occur are substantially different in Denver, Colo., as opposed to Miami, Fla., due to the loss of atmospheric pressure with altitude. It is commonly known, for example, that the temperature at which water boils in Denver is lower than it is at sea level. This can also be important when a patient is being treated with the application of the subatmospheric differential pressure and is being transported from one altitude to a different altitude by ambulance, for example, or by air transportation.

The term “negative pressure” has been used principally as if the pressures mentioned above were some kind of salve or lotion which is sprayed or applied in some way to a wound cavity. Unfortunately, as above fully discussed, “negative pressure” does not factually and realistically exist, and, therefore, it merits no further discussion.

The second term, subatmospheric pressure, is illy defined but is more factually correct, and it can be interpreted in several ways. For instance, the atmospheric ambient pressure at noon today might be 765 mm Hg, 30.11 inches of water, or 1020 millibars, but yesterday at noon it was 760 mm Hg, which is 29.92 inches of water, 1013 millibars. Both of those are atmospheric ambient pressures,just at different times at the same place.

For example, if yesterday at noon, one applied a subatmospheric pressure gradient value of 4 mm Hg (0.19 inches of water, or 6 millibars), the realized subatmospheric pressure would be 756 mm Hg (29.73 inches of water, or 1014 millibars). With that gradient value set in absolute terms, and thus having the 4 mm Hg (0.19 inches of water), it is always a subatmospheric pressure which remains that much lower than ambient atmospheric pressure, whatever the atmospheric ambient pressure value is, or will be, that is being experienced at any later time.

The ambient atmospheric pressure is not constant—it is forever changing under conditions of temperature, elevation above mean sea level, and weather conditions. Several so called “standard pressures” have been defined for one reason or another. The standard ambient atmospheric pressure that is now generally accepted is the absolute air pressure of 29.92 inches Hg at sea level at a standard air temperature of 15 degrees Centigrade.

To apply the term “atmospheric pressure” to wound therapy, one must emphasize that the referenced atmospheric pressure is the ambient atmospheric pressure at the place and the time of the application of the pressure-assisted therapy, and its changes as treatments continue. All other pressures must relate to that value of pressure. The absolute pressure to be imposed within the wound cavity can also be called an air pressure, but it is of a lower pressure value than the referenced ambient atmospheric pressure. Thus, a pressure differential is imposed, which, by itself, means little until it is realized that the pressure differential, which is the atmospheric pressure minus the differential pressure acting upon physical surfaces or through mobile fluids, creates forces which originate from the ambient atmospheric pressure, and these forces can be the engine of healing in this system of wound therapy.

Unfortunately, little, if any, analysis is detailed in the published literature with which the inventors are familiar as to what these forces can do, and they do exert forces, either positively or negatively, at any particular magnitude of force. Are they excessive or unproductive, and at what magnitude, when, and why? How can one grasp the meaning of these forces in terms which can be easily understood? One way is to examine the pressure terms used by the meteorologists in measuring weather phenomenon, and then have a greater appreciation of even a 10% or 15% decrease in the atmospheric pressure (a decrease of the ambient atmospheric pressure, where, with the standard 14.7 p.s.i., from which there are decreases 10% being a decrease of 1.47 pounds to 13.23 p.s.i., or a 15% decrease being to 12.28 p.s.i.) which, with the accompanying winds, water surges, and spun-off tornadoes of hurricanes, creates respect for those forces. A decrease to that extent, once filly grasped, has an awesome meaning.

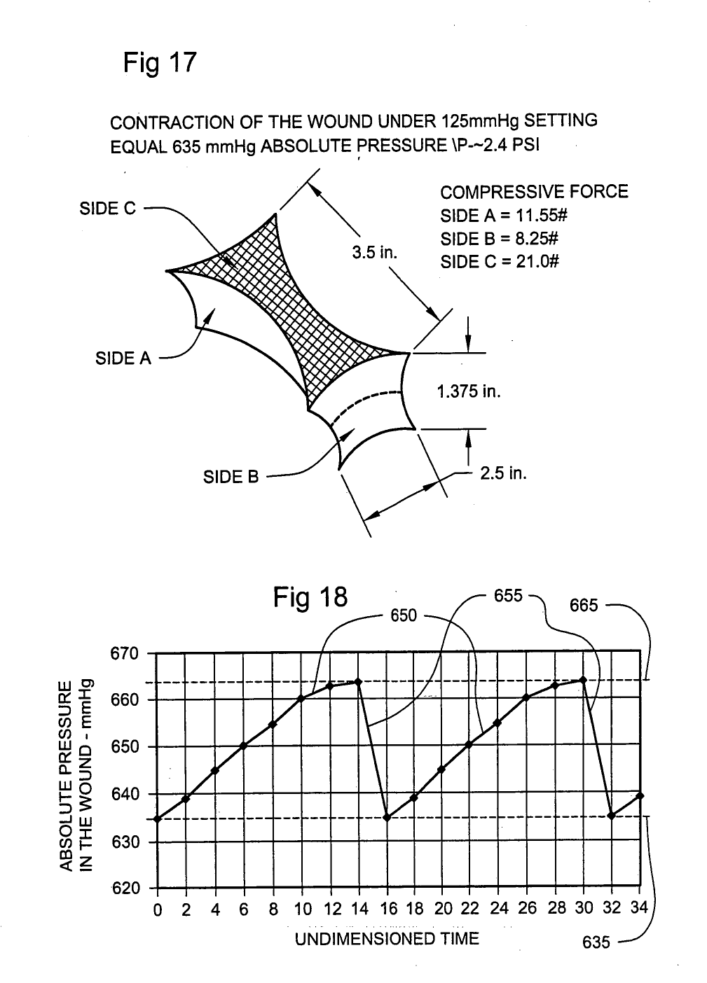

A set differential subatmospheric pressure value of 125 mm Hg, irrespective of the ambient atmospheric pressure, is the ambient atmospheric pressure minus 125 mm Hg of pressure, acting on the wound so that the pressure differential is tending to close the wound cavity because the pressure elsewhere within and outside of the human body (except for the blood pressure) is the same as the ambient atmospheric pressure at all times, whatever the numerical value of that pressure may be. That differential 125 mm Hg pressure equates to 2.42 pounds per square inch. This subatmospheric differential pressure would then be acting against a wound wall having an area measuring 6.45 square inches, and would be applying a force of 2.42×6.45=15.61 pounds. Being a subatmospheric pressure, it tends to cause a collapse of the space within the wound and any dressing therein such as a sponge, by causing the walls of the wound to move toward each other. This is best seen in FIG. 17.

One of the tested wound dressing materials, used for several years by a supplier of a leading pressure-assisted healing system and which is inserted within the wound, is the identically same material that was actually obtained from that supplier, and it has been extensively tested for its maintenance of effective porosity and permeability. That exact material was shown to completely collapse at a 90% reduction in volume upon the application of a pressure of 1.867 pounds per square inch, which is equal to 96.68 mm Hg. Thus, in the example shown above, the entire system would be closed down when subjected to 125 mm Hg due to the loss of effective porosity and permeability of the material that was inserted into the wound. The purveyors of the leading system have recognized this problem, and have gotten around it by inserting certain steps in the operation of the system which closes down the pump that is creating the 125 mm Hg “negative pressure” for a period of time, which results in allowing the “negative pressure” to change so that it approaches the ambient atmospheric pressure in the wound cavity containing the wound dressing material noted for a period of time, and then turning the pump on again so that it again is generating the “negative pressure” within the wound. This system was given the name “Intermittent Operation” and it is a mechanical arrangement which has a predetermined cycle of producing the subatmospheric pressure to the wound stopping and restarting the pump that generates the subatmospheric pressure each time that a high differential subatmospheric pressure is reached, and only after a very low pressure has been reached is the motor restarted. In the example of the paragraph above, the subatmospheric differential pressure would have to be less than 96.68 mm Hg (1.867 PSI) for the system using that particular material to operate with even when it was reduced to 10% of its unpressured volume, and probably a good bit even less in order to be reasonably effective with the pressure supply being continuous at such a considerably lower subatmospheric differential pressure level.

Observation has indicated that it seems to be standard practice for the medical personnel to carefully measure the length, width, and depth of a wound in centimeters during each change of dressing on the wound, and record these details for the record. There have even been highly technical ways to obtain these measurements, including the volume of the wound when there is only ambient atmospheric pressure involved. Some of these ways could be easily used to obtain the data that is put into the computer program. However, in the current practice with the current equipment on the market by KCI™, Blue Sky™, and some others, that information is seemingly not thereafter put to any use. Since there are no instructions from the maker or seller of the devices, it appears that all of that data are just being ignored or forgotten as if they have no further use. It certainly is not used to fine tune the details of the future course of the therapy after any changing of the dressing. Additionally, the critical properties of the wound dressing material are in no way considered in the selection of changing therapy parameters because there are little or no instructions provided by the maker or seller even remotely relating to the consideration of those critical properties by anyone who is in the chain of production, sales and use. The same is true even if the machines with which the dressings are used are being leased or otherwise rented by the users instead of being sold or in any other manner provided to them for their use.

It is an important feature of the invention herein disclosed that several considerations need to be given in order to select the best available wound dressing material for the wound therapy. It has appeared that very few, if any, such considerations have been given in selecting and using the wound dressing material that is inserted into the wound. These considerations include defining the various properties of that material which are required for each part of the entire dressing, or are at least found to be very desirable. At a minimum, several of these material properties should be required, and they must be used if the users of the invention herein disclosed is to obtain good results. Even more of these material properties should be used if the best results are to be attained. Of course, there may be some properties that are desirable but which are not necessary, and which would not be an economic benefit should that feature be used at this time. The use of these properties which are either required or are highly desired is within the purview of the disclosed invention.

There are several properties of the wound dressing material that are of critical or desirable importance, and which, along with wound dimensions, should dictate the parameters of the system and method of therapy. These properties are as follows, where the stress applied is a differential pressure or a mechanical force acting across an area:
I. Porosity and its change under stress.
II. Permeability and its change under stress.
III. Compressibility and the resultant change in volume under stress as the material is being compressed.
IV. Structural Integrity and the characteristic of its resilience reaction to repeated stress caused by compression and release actions acting on the material.
V. Pore size distribution and its change under stress.
VI. Bioactivity reactions, if any and the extent thereof, with bodily fluids and tissue.
VII. Hydrophobic and Hydrophilic properties
VIII. Tensile Strength, including the ability to resist tearing when pulled.
IX. Shape of the wound dressing to be inserted into the wound in relation to the shape of the wound.
X. Pore surface area of each member that is to be inserted into a particular wound.

This is closely related to the bioactivity property. While this is not directly needed for wound dressing selection, the amount of such surface areas needed for bioactivity reactions is of importance because of its effects on the functioning of the member as it is stressed by being compressed and released.
XI. Permeability. This is not needed for selection of a particular wound dressing, but should be measured so that this property is known and the functions of it can then be accurately determined and controlled when appropriate.
XII. Material Density. This property is also not a determining factor for selection, but is easily measured.

Each of these properties will be examined in more detail in the following text and initial specification ranges will be delineated in the search for the ideal wound dressing material. Many of the properties are interrelated, and changes in one will result in changes in one or more of the others. Details of the required tests will also be discussed

The property of POROSITY: Porosity is defined as the percent of a particular volume of a material which is open space. There are two kinds of porosity: Total Porosity and Effective Porosity. Total Porosity is the percent of a particular volume of material which is open space. Effective Porosity is that percent of a particular volume which is open space and which is interconnected and available for the transmission of fluids through the material upon the imposition of a pressure drop in the fluid medium.

Forces which compress a particular volume of wound dressing material will affect the Effective Porosity, the Permeability, the Pore Size Distribution, and the Shape of the wound dressing. The minimum Effective Porosity retained by the wound dressing material placed in the wound, when the wound dressing material is in place in a wound and is submitted to the system's maximum allowable subatmospheric differential pressure, should be at least approximately 20% of that material's Total Effective Porosity when it is not under any other stress. The Effective Porosity of a dressing material should never be reduced to essentially zero percent, which will occur when there is complete collapse of porosity of the material under sufficient stress. This would render completely ineffective the ability of the wound care system to perform its function, and could, if not quickly detected, cause damage to the wound area that will make it at least very difficult to assist in the healing process, or even very negatively affect the healing process.

PERMEABILITY: Permeability is the ability of a volume of wound dressing material to transmit a fluid (in this case usually a combination of air and some liquid) through its Effective Porosity by the imposition of a pressure drop in the flowing fluid, usually but not always a liquid and air mixture, from one side of the wound dressing material to the other. This ability is measured in Darcys or MilliDarcys. Permeability is affected by any change to the Effective Porosity. For a given set of stressed wound dressing specimens, as specified above for the porosity tests, it can be measured by the same equipment as specified above. The level of Permeability should not be less than 50 milliDarcys at 80% compression of the wound dressing material.

COMPRESSIBILITY: For the purpose of specifying a wound dressing material, Compressibility is the percent loss of volume for a given level of stress. The loss of volume will affect the porosity, the permeability, the pore size distribution, the shape, and possibly the structural integrity. As shown in FIG. 14, a standard size of sample that has been selected for these tests was 6 cm.×6 cm.×3.5 cm., with the stressing force being applied to the side measuring 6 cm.×6 cm. Effective porosity and permeability should be evaluated for each level of stress selected. Special attention should be given for the stress level at which porosity and permeability will have been effectively eliminated. A special testing device, designed and constructed to make the measurements of the actual porosity and the actual permeability of the wound dressing material is desirable while that material is being used as a part of the system, and these measurements should also be recorded. If that is not done, then the material or materials for using this material should be so tested before it is approved for use in a system, and they should support data which indicate that the material or materials tested pass these tests. These materials would employed in the system and particularly in the wound dressing mentioned above, so that the Effective Porosity under porosity is an integral part of the setup to attain satisfactory performance of the entire system. Maximum volume loss of at least 80% to 90% should delineate the point where porosity and permeability are essentially extinguished, and so long as that volume loss is not attained, the system would not fail because of excessive volume loss of the material that is a part of the wound dressing.

The property of STRUCTURAL INTEGRITY, for the purpose of specifying a wound dressing material can be characterized as the resilience of the core material under repeated stress. The standard test may be as follows: on the 6×6×3.5 cm. test sample defined above, the testing device should compress the sample (with force applied to the 6 cm×6 cm. face) to the point where porosity and permeability are distinguished (as defined by the test above) for a repeated cycle of 500 compressions during a 48-hour period, The lowest percent loss of volume when material being tested returns to the unstressed state after tests is to be the measure of the optimum acceptability as a wound dressing material, and the minimum acceptability of a tested material should be a percent loss of volume of no less than 80% to 90% when the point of the porosity and permeability is extinguished. If a wound dressing material, when tested, reaches that point where porosity and permeability is extinguished and the percent loss of volume is substantially less that 80%, that material should not be accepted for use in a wound dressing environment.

The property of Pore Size Distribution is the correlation between the percent of pores in a given sample of wound dressing material which are less than a particular pore size expressed in microns. Pore Size Distribution is inherent in all man-made or natural porous materials. The property is important as it is affected by the initial Effective Porosity, Compressibility and Structural Integrity, and this property must be evaluated at the selected stress levels on the standard sample above. In addition to the changes in Pore Size Distribution accompanying changes in Effective Porosity and Permeability, this property is of vital importance to the design of a wound dressing material which, at all levels of stress to which it can be exposed when treating a wound (but not necessarily at its unstressed state) will exhibit a minimal number of pores of a size into which granulating healing cells of the body could intrude—thereby rendering the removal of the wound dressing material either painful to the patent being treated or possibly hindering the healing process. As an initial practical target, the first level of stress should, as completely as possible, extinguish all pores of a size larger than 100 microns if the material has such larger sizes when in its unstressed state.

The property of BIOACTIVITY is what, if any, chemical reaction occurs, including the extent to which there is chemical reaction of the wound dressing material with anything with which that material has been treated in relation to the bodily fluids or tissue with which it may come into contact. This property may not be emphasized by a buyer during the initial selection of a wound dressing material to be used, but it may become of prime importance if the enhanced healing capability is considered. Any sample which is, or will be treated, must be evaluated, utilizing the standard tests detailed above as a potential wound dressing material in both the treated and the un-treated states.

HYDROPHOBIC AND HYDROPHILIC properties, though of some basic interest, are not so important unless the BIOACTIVE property may require either of these two properties to be active. This is not considered to be likely based on the fact that the range of absolute subatmospheric pressures to be created in the wound cavities would not be of a magnitude to vaporize water at the normal temperature of the human body, and the permeability at any one stress level would not be affected by the sample being of either Hydrophobic or Hydrophilic.

The property of TENSILE STRENGTH is defined as that amount of force applied to the wound dressing material which would cause the material to tear apart when it is being removed from the wound. An appropriate test can be made using a sample of the wound dressing material which is 10 cm in length×4 square cm cross-sectional area. One end must be secured, and the other subjected to a tension force tending to tear apart the sample. The recorded force at which the sample fails would be the indicator of its acceptance or rejection. An initial indicator of 3 to 4 pounds of applied force would be the minimum acceptable.

The SHAPE OF THE WOUND DRESSING as it is inserted into any one wound is extremely important. The reasons for this will be covered more thoroughly elsewhere in this patent specification. Simplified, it must reasonably fit, in cross-section, the shape of the wound.

There are some other dressing material properties that are not related to the selection of a satisfactory wound dressing material unless there are one or more of the above-noted properties which would require them to be considered. These include the following additional properties.

PORE SURFACE AREA. This property, while it is of importance in the application of Bioactive materials, and so long as the material is not Bioactive as above defined, it is not a determining factor in the selection of a wound dressing material. However, it should be routinely measured so that if there should be some change to the material that makes it at least questionably Bioactive for wound care purposes, the base information for the material before such a change would be on record. After all, once the wound dressing is manufactured and packaged, one must consider the possibility that it will be used together with-the application of Bioactive materials.

GAS PERMEABILITY. Since the parameters of wound therapy utilizing the CAPE™ modality do not envision the occurrence of gas in the wound cavity, this property is not important in the selection of a wound dressing material, but should be measured, and can be used as a defining property in the selection of a wound dressing material instead of liquid permeability.

DENSITY of the material. This would not be a determining factor in the selection of a wound dressing material, except that the property should be routinely measured while other characteristics are measured.

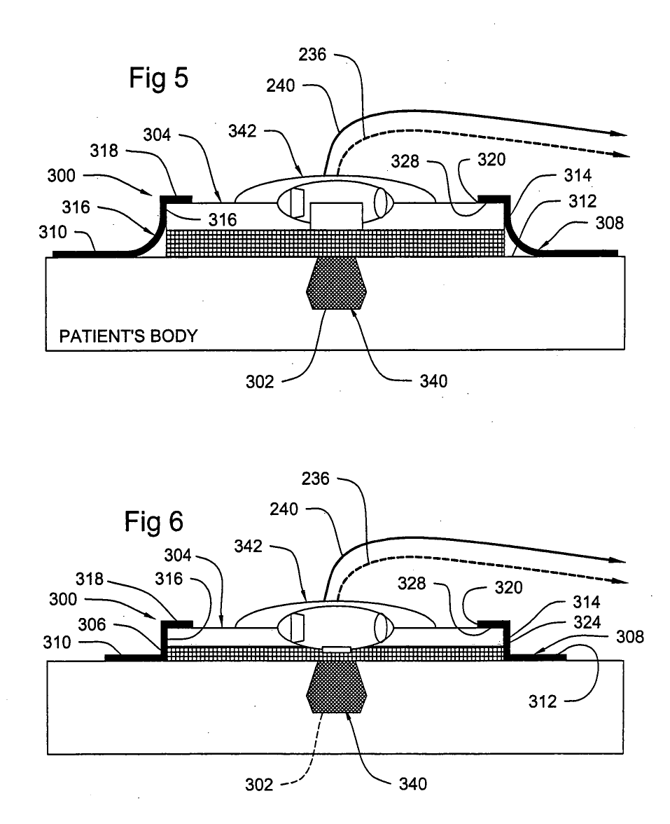

The clarification of the basic physics discussed and the selection of a wound dressing material exhibiting optimum values of the properties shown above comprise the basis of a new and novel wound therapy system where the medical practitioner will be able to exercise full control over wound healing utilizing a gravimetrically generated absolute pressure within the wound cavity. The examination of the properties above will also enable the selection of the optimum material for the “Sealing Blanket” discussed above and shown in FIGS. 5 through 9 of the drawings of this patent application.

### Detailed Definition of Terms Used In Subatmospheric Differential Pressure Healing

Force is the motive engine tending to drive an object in a particular direction due to the imposition of a pressure differential on the receiving area, and across the object or fluid. Its units are Pressure times Area equals Force.

Pressure: In the realm which is of interest to the healing process, pressures are all referenced to the force per unit of area imposed by a column of air in the atmosphere at the time and the place of the measurement. The absolute atmospheric pressure is the column height of a material, engaging a specified area of the bottom end of the column, which will balance the atmospheric column, and is measured in millimeters of mercury, inches of water, or some other standard, depending upon the standard most familiar to the users. In the English system, most users are familiar with the pounds per square inch (p.s.i.) measures, and the English system of distances and areas, and in the medical field, with millimeters of mercury (mm Hg) and the metric system of distances and areas. It should be made clear that the column of air, mercury, water, etc. can be of any aerial size (i.e. one square inch, 10,000 square inches, one millimeter, 5,000 square meters, one square centimeter or 30,000 square. centimeters, etc.) and the column height of any substance which will balance the pressure exerted by the column of air in the atmosphere will indicate a measure of atmospheric pressure in all cases. Obviously, the area of the device doing the measuring also does not affect the pressure reading, and it then follows that, at any one time, atmospheric pressure is, for all practical purposes, constant over the areas of interest in wound healing, and, as this application is based on the absolute value of a “Differential Subatmospheric Pressure” referenced to the ambient atmospheric pressure at any time, the absolute value of a selected Differential Subatmospheric Pressure setting will remain constant (except for control cycling, as described in detail below) regardless of the momentary value of the ambient atmospheric pressure.

Permeability and its Basis: In 1856, Henry Philibert Gaspard Darcy first developed the equation to describe fluid flow through a porous medium:
Q=−(kA/μ)*(dP/dx)

Where:
Q=Volumetric Flow (cm³/second)
k=permeability (Darcys, (cm²*dP)/(seconds*atm))
A=the cross-sectional area of the porous media (cm²)
μ=viscosity of the fluid (centipoises)
P=pressure (atm) (dP=the pressure drop across the media[atm]. Darcy considered this a negative quantity, hence the minus sign in the equation.
X=the length of the flow path through the medium (cm)
dP/dx=the pressure drop (atmospheres) per centimeter of flow path.

Using the above equation, it is possible to determine the permeability (k) of the porous media. This property is most important where the fluid being transmitted is valuable and great quantities are needed in short times, but it is also extremely important where some small positive amount of fluid transmission is vitally important at all times.

There are four conditions that are required for this equation to be valid:
1. Creeping flow regime—The Reynolds number based on superficial velocity must be on the order of 1. This value is applicable in differential subatmospheric pressure wound healing.
2. The porous media is not chemically reactive with the flowing fluid.
3. There is no static accumulation of fluid in the pores of the media.
4. There must be only single-phase fluid flow.

Hydrophobic (water repelling) and Hydrophilic (water attracting) Properties do not enter into the situation. In certain cases, these properties are important, and they affect the ability of the body of material to transmit fluids when a differential subatmospheric pressure is imposed. They are absolutely important when there is more than one fluid being transmitted, in which case a field of physics called “Relative Permeability” must be considered. Fortunately, in the differential subatmospheric pressure wound treatment field, only one fluid (liquid) is being transmitted, and a differential subatmospheric pressure of some 710 mm Hg (a differential subatmospheric pressure which would never be used) would have to be imposed to cause the wound/wound dressing to contain two fluids (a gas and a liquid). This is due to the fact that water will only vaporize at the temperature of the human body at an approximate absolute pressure of 50 mm Hg.

There is a certain misconception in the medical literature that a more “dense” wound dressing material causes the need for a higher differential subatmospheric pressure for the full application of the subatmospheric pressure treatment. This is in error. The application of full differential subatmospheric pressure to the wound would take only seconds with a wound dressing with a certain permeability, and might take one second longer with a wound dressing material which has only one tenth of the permeability of the first sample. The difference is time only—and that small difference is insignificant.

### Basic Differential Subatmospheric Pressure in Wound Healing

A. Movement of Fluids by pressure. Fluids (gases and liquids) only move by the imposition of a differential pressure oriented in the required direction of fluid flow, and only then if the liquids/gases are not completely obstructed in their path from the larger to the lesser pressure. (i.e., the “effective porosity” is positive—not “0%”.)

B. Pressure-generated forces. Solids, liquids, and gases move by the imposition of a force oriented in the direction of the required movement. The impetus to move is determined by the pressure applied to the area exposed to the higher component of the differential pressure which results in a force which is equal to the area exposed to the larger of the pressure components multiplied by the pressure imposed upon this area, and this force is resisted in the opposite direction by the lesser of the differential pressure components multiplied by the area exposed to the lesser component. The magnitude of the net force (larger force minus the weaker force) is the driving force resulting from the imposition of differential pressures, and this is the operative force in differential subatmospheric pressure-assisted wound healing.

C. The term “Negative Pressure.” It is used to mean some pressure that is less than a pressure from which it is measured.

D. Pressures in the human body. The human body contains throughout it the normal ambient atmospheric pressure to which it is exposed, except in the blood circulating system. Otherwise, ambient atmospheric pressure would simply collapse the body and blood would not flow. Blood pressure is a measure of pressures always somewhat above the ambient atmospheric pressure. Systolic Pressure is the value measured at which the sound of blood coursing through the arteries ceases due to the pressure exerted by the blood pressure cuff, and Diastolic Pressure is the value of pressure at which the sound of the blood pulsing through the vessels subsides. It follows then that there is already a differential pressure (however weak it may be) tending to close wounds and it is caused by normal blood pressure vs. the pressure in the wound at the time the wound occurs, and thereafter if there is no other intervention.

E. Process Control in the relatively small volume of wounds. It is easy to see that, if a certain differential pressure is imposed on a healing wound and bodily fluids are entering the wound, the absolute pressure within the wound would rise, and the differential pressure would start subsiding. If the wound/system were connected to a very large pressure system, this change in the small wound volume would have only an infinitesimal effect on the total volume of the large pressure system. However, in the relatively small volume of the differential subatmospheric pressure system/wound, means must be provided to maintain the required differential subatmospheric pressure value as constant as possible as bodily fluids enter the wound cavity. Such means of control, necessarily cause the absolute subatmospheric pressure within the wound cavity to cycle between the upper and lower pressure control points imposed by the control mechanism, meaning that the pressures used in treatment may be Cyclic, but not Intermittent. The cyclic use of pressure according to this invention is not a predetermined sine wave type of cycles. It is a particular set differential subatmospheric pressure that can be modified to a slightly different differential subatmospheric pressure when conditions warrant that change. That modified differential subatmospheric pressure is then the pressure that is applied to the wound. If no modification occurs in a treatment session, then the applied differential subatmospheric pressure is constant and not cyclic. Intermittent application of differential subatmospheric pressure occurs when the differential subatmospheric pressure is allowed to subside between cycles to “0” pressure. A further means of control in such cases is to allow unfiltered air to enter the system at the point of lower absolute pressure such that the differential subatmospheric pressure remains almost constant. There are two situations, one not being in favor of the use of this system, but nevertheless may be used, and if it is used, it still will be subject to the invention herein disclosed and claimed. The one not particularly desired is the introduction of air into the system which may, itself, contain contaminates because of poor or even no filtration. Of course, such air bleeding into the differential subatmospheric pressure being produced to reduce the differential amount would normally flow away from the wound, through the pump and back into the ambient atmosphere. To completely assure that there can be no contamination after the pump is no longer running, the air that is to be introduced into the system should be highly filtered, in case it should at any time be able to infiltrate any part of the system. The other is that it requires the pump to operate continuously, at least as long as the treatment session lasts. This is a preferred usage, it being understood that the pump must then be capable of having its “output” differential subatmospheric pressure modified at times. This is preferably done by controlling the speed of the impeller in the pump. This requires larger, or at least more powerful and thus usually much more expensive, batteries if the system is to operate as a portable unit without access to an outside source of electrical power. Yet, it is important at times to use the system of the invention as a portable unit. One such occasion is when the outside source of power fails, and the other occasion is when there is no outside source of power available to which the system may be connected.

F. Shape of the wound dressings. The dimensions of a particular wound are measured (to the extent possible) in length, width, and depth in centimeters. As few, if any, wounds result in a perfectly shaped “box” or triangular cross-section, the wounds must be measured as closely as possible with the above dimensions and at least each 48 hours. It has been shown in the medical literature that normal wounds, when healing is progressing, will exhibit an exponential decline in volume and/or surface area with time. The wound dressing itself must be shaped to fit the wound as closely as possible, to completely embrace the wound walls, and of a shape and with the requisite properties that will resist over-collapse (with consequent extinction of both porosity and permeability) upon the imposition of the positive differential pressure. It must be changed regularly because its use over the wound usually involves some interstitial fluids from the wound, and also because of the decrease in size of the wound as it heals. Over-collapse is defined as the wound dressing collapsing to an extent greater than the wound under the same differential subatmospheric pressure such as to leave areas of the wound walls un-embraced by the wound dressing material. The shape of a part of the wound dressing that is inserted into the wound should always be with tapered sides which will result appropriately directed force vectors to assure that the wound dressing will maintain substantially full contact at all times with the entire wound wall.

### Method of Operation of the Device and the System

The invention includes devices and a system for differential subatmospheric pressure assistance in the healing of wounds on the human body and the method of healing wounds using the same devices and system. The differential subatmospheric pressure is always referenced to the ambient atmospheric pressure existing during the time of the treatment. While at any one location there is usually very little if any change in the ambient atmospheric pressure, there are very definite changes if the location is in a flying hospital-like transport, or even in a vehicle that is driving from one altitude to another some hundreds or thousands of feet higher or lower. The inventive device contains means with which to establish the required differential subatmospheric pressure, to measure the magnitude of the differential subatmospheric pressure in the wound at any time, to maintain the magnitude of the differential subatmospheric pressure within imposed limits, to measure the temperature within the wound being treated, to record the instantaneous data being measured, to measure other parameters relating to the wound and its treatment, and to convert all of this data to computer files recorded on electronic devices which can later be transferred to a computer for detailed examination, to allow the input of the measured dimensions of the wound to be treated, to select one of a plurality, and usually four, different modes of treatment, i.e. “Aggressive, Relaxed, Soft, or Gentle” or any other word labels that will connote the differences in each mode, and the means to design and insert a wound dressing into the wound with the proper shape and possessing the requisite physical properties to augment the healing process as treatment progresses. It is desirable that it provide means to withdraw and measure any fluids expelled by the body during the healing process, and to allow means of sampling any expelled fluids to test for unwanted components. The main purpose of the device and the system is to allow the medical practitioner to exert maximum supervisory control over the details and progress in the healing of a wound, while the routine of considering all facets that may normally occur which should cause the applied differential subatmospheric pressure to be either increased or decreased to a limited extent by use of a computer and a computer program and a subatmospheric pressure source, such as what is known as a vacuum pump, to control and modify the actual differential subatmospheric pressure applied to the wound to be changed accordingly, and having such modification to occur as the computer using the computer program directs. At the same time, the control supervisor of the treatment does have the ability to manually overcome computer-set differential subatmospheric pressure value when needed. Such a need could occur should there become some unauthorized leakage of ambient atmospheric air into any part of the system that has the differential subatmospheric pressure in it. This could be leakage where the dressing is to be sealed relative to the tissue surrounding the wound, or a leaking or ruptured tube or the connections that connect the tube either to the dressing or to the pump. It could also occur if the pump ceases to work, or speeds up and produces a much changed differential subatmospheric pressure. The system and method of the invention is able to help the nurses and physicians to best utilize and to facilitate the understanding of the basic physical laws which govern the interrelated forces being employed to assist in the healing of the wound and to use the data obtained to design even better specific therapy for future wound treatments.

The computer programs that can be used to practice the invention show the following information, and provides for the entry of information:

1. Name of the patient to be treated.
If already a patient, then ask for his record.

2. Select the Mode of the treatment session.
This means to select one of the following:
   Aggressive (the highest differential subatmospheric pressure)
   Relaxed (a lesser differential subatmospheric pressure)
   Soft (a still lesser differential subatmospheric pressure)
   Gentle (The lowest differential subatmospheric pressure that we can effectively use)

3. Enter the dimensions of the wound to be treated, in cm:
L—length______ W—width______ & D—depth______

There will be an opportunity presented to accept the values entered, or to reject them and enter corrected values.

4. There may be an information screen, warning that the system will default to the properties of the CAPE™ wound dressing material selected by the rigorous tests noted herein, and the use of other materials that are considered to be contra-indicated, and may be dangerous to the patient's health.

5. There will be a summary of the impending treatment session, showing at least some of the following. Some of this information may be optional, however, such as those having an “O” in front:
Name of the patient.
Name of the Mode selected for the treatment session.
(O) Use of the wound cover blanket only, or the blanket and the wound dressing insert.
The dimensions of the wound: L, W, & D.
(O) The largest area of the wound to be exposed to the selected differential subatmospheric pressure.
The pressure control setting to be sent to the pump from the computer, based on the entered data. Can be in mm Hg, or other choice of measurement.
(O) The expected compression percent of the insert to be used in the treatment session.
(O) The expected reduction in porosity of the insert for the treatment session.
(O) The expected reduction in the permeability of the insert for the treatment session.
If all of the information and data above is acceptable by the operator, then enter “a” for acceptable, if not, then enter “n” and start over.

6. If accepted, there may be a screen that asks the operator to confirm that certain procedures have been (or will be) performed, and data entered. These points to be confirmed are:
The dressing is applied and shaped to fit the wound.
The seal blanket is applied and is sealable or sealed.
All data and sample lines are attached where gathered and where data and samples are to be sent.
The power supply is 110 volts AC, or
The power supply is the backup battery.
All variables concerning this patient have been selected.
As the system starts, it appears to not cause the patient distress.
You will be checking the patient regularly for distress while system is operating.
You will be checking on various readings regularly, and will be on alert if the system sounds an alarm.
Yes: ______ or No: ______. If no, then system preparation process will restart.

7. While the system is running and the treatment therapy is in process, the following information will be continually received, and be updated:
Your therapy is now in progress.
Current wound temperature is ______ degrees F.
Current differential subatmospheric pressure in the wound is: ______ mm Hg. (or other unit measurement being used)
Current time is (day) (month) (year) (time in hours, minutes, seconds, and what time zone applies. (Order may differ, depending on desire)
Elapsed time of this session is ______ hours, ______ minutes, ______ seconds

There will be a place here to stop the treatment. It may be labeled appropriately such as “Quit Session.” Choosing that will stop the session immediately.

Depending upon the proprietary computer program being used, the treatment the computer gives to the data may be processed in different manners. One applies the data and reaches the ultimate issue of whether or not to change the currently set differential subatmospheric pressure using formulas that may include logarithmic calculations, while another one may use simple straight-line calculations that involve only additions, subtractions and straightforward multiplications, as well as the value that each sensed condition has in relation to the changing of the set differential subatmospheric pressure being used. By way of example, the temperature increase of 1° F. above normal may be sufficiently large to increase the differential subatmospheric pressure by a certain amount, and that would be recommended, assuming that none of the other data would result in a change of that pressure. Some other sensed condition may not need to recommend a change in the differential subatmospheric pressure until it has had a major change. If several data would recommend different changes in that pressure, based on each sources “value” for that purpose, then the recommendations for changes would be algebraically added, and the net change would be the one recommended, sent to the pump control, and actually changed. Either type will be satisfactory. The proprietary program originally considered is of the first type. The other one, a more simple approach, was then developed.

### Advantages of the System Over Prior Art

The device and system of the invention contain a proprietary computer program that is an integral part of the invention herein disclosed. It gives the medical practitioner an array of conditions from which to select for healing the wound under consideration. Once set and started, the system modifies the differential subatmospheric pressure in the wound when one or more sensed conditions merit such a change to maintain a desirable differential subatmospheric pressure in the wound. It does not have to wait until someone recognizes the need to make such a modification, and then manually does so. Further, the data from the healing wound, and any change of conditions, will be collected, recorded, and made available on magnetic or some other suitable media such as DVDs and CD-Roms, and others that now or later become available by or on which to save data for later analysis. This is leading to ever more sensitive and accurate therapy in the future instead of using guesswork or hunches as to the amount of subatmospheric pressure to be applied to the wound, for example. The use of that computer program is disclosed in more detail elsewhere in this patent specification.

The inventive system and method herein disclosed contain control mechanisms which are to be set by computer control by the health practitioner inputting the requested data about the wound and the patient, and engaging sensors to sense and send data on certain conditions in and relating to the wound being treated, and having a computer output that sets the target subatmospheric pressure to be delivered to the wound by controlling the differential subatmospheric air pressure from a source of that air pressure. The program provides its proposed subatmospheric pressure to be used to confirm or to modify the differential subatmospheric pressure being delivered to the wound, with a provision to override the settings of the computer at the behest of the practitioner before or very shortly after the computer actually sends the solution to the subatmospheric pressure generator control, such generator being what is commonly known as a vacuum pump. If the source is a systemic outlet from a larger subatmospheric pressure generator as is often provided in hospitals, it will send its solution to a pressure modification and control mechanism so that the pressure actually delivered to the wound is that which the computer solution gave. It is to be understood that, at times when there is reference to the pump, it also encompasses such a mechanism when a distribution source of subatmospheric pressure is used, and vice versa.

The inventive system contains hardware devices for the collection of data on what is happening in the wound, and it is not dependent upon devices and/or materials designed such that they may become plugged or disabled by the healing process, or designs which may spread contamination during operations.

The system also contains fluid collection and testing devices which allow problem-free fluid collection and interstitial fluid sampling for the testing for contaminants in the bodily fluids.

The system further contains means to record data concerning the amounts and the rates of expulsion of expelled bodily fluids, the concentration of other materials than the fluids themselves, usually having their source being the wound and the tissue defining it.

The system also includes a new and novel wound dressing having a blanket seal which utilizes the differential subatmospheric pressure imposed on the wound and present on the inner or wound-covering side of it, together with the ambient atmospheric pressure on the outer side of it, to produce forces that cause a seal action which is maintained in engagement with the patient's body section that is adjacent to and encircles the walls of the wound that does not depend upon the use of adhesives which can, and will, often cause great discomfort and, at times, possible injury to the patient upon removal thereof. This wound dressing also utilizes a technically much more desirable porous and permeable insert placed into the wound, unless the wound is a shallow wound, and having the optimum porosity, tensile strength, pore size distribution, permeability, structural integrity, chemical reactivity and shape in cross-section and length to produce a sponge-like insertion that fits in the wound. The air being removed from the wound by the differential subatmospheric pressure passes through this insert.

The invention also includes means to set an upper limit for operating differential subatmospheric pressures which will allow continuous “cyclic” operations within a preset range of differential subatmospheric pressures during healing—not the so-called “intermittent” operation, which is really a start-stop-start-stop operation where the differential pressure that is put out by the vacuum pump is allowed to subside to or near to the ambient atmospheric pressure and to then start up and pump at a relatively high delivery differential pressure until the upper value limit for the pressure within the wound is reached, and then again the vacuum pump stops delivering any subatmospheric pressure. The range of these limits will be set such that the safe maximum usable pressure range of the differential subatmospheric pressure being applied to and present at the wound can be used when needed without endangering the patient as has happened at times with the presently marketed system, and the pressure being used at any time can be held constant to within the very smallest fraction of the imposed differential subatmospheric pressure possible with modern technology. At the same time, that pressure may be modified to be more or less differential subatmospheric pressure in accordance with changes in and of the wound in response to sensor-generated signals that are transmitted to a computer controlling mechanism that compares the needs of each item being sensed in relation to each other and to the differential subatmospheric pressure in the wound, and orders changes as needed to the applied differential subatmospheric pressure. This maintenance of substantially precisely the desired differential subatmospheric pressure to the wound instead ofpermitting it to cycle over a relatively much wider range of such pressure as occurs in the present commercial systems of this type is preferable. It is much better for the wound to always have the proper differential subatmospheric pressure in it than for that pressure to vary widely back and forth from more to less of the differential subatmospheric pressure being applied. It is much like having a substantially constant controlled atmospheric temperature range on the moon, where the average temperature over a lunar month may be somewhat livable if not comfortable, but the extremes are such that human being could not survive either the heat or the cold.

As a part of the most preferred version of the system of the invention, and also as an invention that could stand alone, there can be used the innovative wound dressing materials to be inserted into the wound during healing which will have undergone rigorous testing of the properties of porosity, tensile strength, pore size distribution, permeability, structural integrity, compressibility, and chemical reactivity. This is a desired but not absolutely necessary part of the invention, and the invention is sufficiently broad to cover either instance.

Within the system of the invention when it has the wound dressing materials noted above, it further includes wound dressing materials produced for actual medical use which are selected, as above, and will be preshaped such that it has sides with downward (away from atmospheric pressure, and outward larger that the surface dimension) sloped sides so that its use will induce vertical downward directed force vectors holding the wound dressing firmly in place during differential pressure healing procedures as well as resisting the deformation that can occur with similar currently used materials that do not adequately conform to the sides of the wound, thus having a tendency to cause tunnels or fistulas to be created that delay the complete healing of the wound for many weeks.

The control center of the inventive system includes devices that gather, use and then store the data concerning the wound's real time condition. The data storage device or devices can employ magnetic media such as currently used discs, hard drives, flash drives, and such or can employ more advanced media such as CDs, DVDs and whatever media that may at the time be available for use now or later. Such media can later be used to download the data into another computer for clinical study of the results to enhance the veracity of treatments in the future, as well as to provide records of the treatment when there is any question at to the competent practice of medicine should the wound treatment not respond in a manner that a patient or some member of his/her family think it should have responded. The control center also may have devices which will display the collected data for the treatment period in progress so that the medical practitioner can see, in real time, the processes and the progress, or lack thereof, then taking place.

### Detailed Description of the Drawings

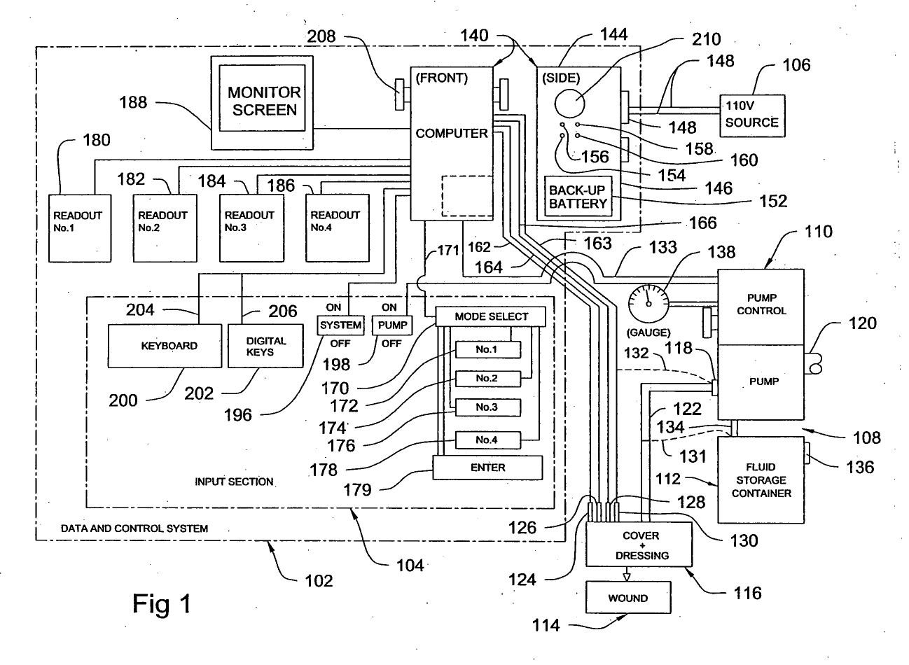

The wound care system 100 embodying the invention is schematically illustrated in FIG. 1. It has a data and control system 102 shown as being bounded by dot-dash lines. Within system 102 is an input section 104 also shown as being bounded by dot-dash lines. The data and control system is the control center of the wound care system. Since these depictions are schematic in nature, and do not show a photographic-like vision, it is to be understood that the various parts of the actual computer center are not necessarily illustrated as a realistic physical embodiment, but are shown primarily to illustrate that the identified parts are there as a part of the control center, and in its most preferred mode all of its components, other than the source of electrical power and, at times, the location of the fluid storage container, are in the computer case. However, for purposes other than having a compact system unit, the computer center, including the data and control system, may be provided so that there is more that one unit comprising the entire system, and would still be within the scope of the invention.

A source of electrical power 106 is located outside of the data and control system 102. This source of electrical power 106 normally provides the electrical power for the entire system 100. The pump assembly 108 and its related items includes the pump control 110, which is really a part of the data and control system, but because it is not necessarily physically integral with that system it is shown with the device that it controls. The pump assembly 108 has several parts, not shown, such as an impeller, a variable speed motor driving the impeller, and a separator that separates the liquid fluid and any particles suspended in it from the air being removed from the wound. Also connected with the pump 108 is a fluid storage container 112. This is the container that receives the liquid fluid and any particles of the wound that it may have in it after it is removed from the wound. The separator that separates the fluid from the air flow from the wound to the pump impeller is located so that the liquid fluid and the particles suspended in it do not pass through the pump impeller and back into the atmosphere. Such separator devices are well known, and have been used for many years, not only in wound care systems but also in other systems where a flow of air will have a liquid fluid that also often has some particles suspended in it.

The wound 114 that is to be or is being treated is schematically shown here, but is shown in greater detail in some other drawing FIGURES. It has a wound dressing and a cover for that dressing, schematically shown in FIG. 1 as 116. The pump assembly 108 is what is commonly called a vacuum pump. It has an intake 118 and an air discharge 120. A tube 122 connects the pump intake 118 with the wound dressing and cover 116, which covers the wound in sealing relation so that the differential subatmospheric pressure being created by the pump is also connected to the interior of the wound 114. This is better shown in some other drawing FIGURES. The wound dressing and cover 116 has a plurality of sensors connected so that these sensors can sense different conditions relating to the wound 114. Four such sensors 124, 126, 128, and 130 are schematically shown. While there are two of the sensors that are the more important sensors, with sensor 124 sensing the temperature in the wound and sensor 126 sensing the differential subatmospheric pressure that is actually within the wound, there may be other wound conditions that are to be sensed, and these are shown as sensors 128 and 130. Additional sensors which may or may not be sensors 128 and 130 may be used, depending upon the particular data that may be desired. For the purpose of minimizing drawing clutter, sensors 128 and 130 are shown as alternatively connected elsewhere. It is to be understood that there may be sensors other than the sensors 124, 126, 128, and 130. Sensor 128 may be alternatively connected as shown by dashed line 131 through the fluid connection 134 where the now separated liquid fluid and any particles suspended therein are conducted into the fluid storage container 112, and may measure the amount of usable storage space remaining in that container. It is usually arranged to sense when the container is about 90% or so full, and when that occurs, it generates a signal that is sent to a part of the data and control system identified later below. This signal may be such things as a flashing light that will draw the attention of the person supervising the treatment session. It may also activate an audible signal, not shown. The container 112 also has a gauge 136 that indicates the container's fluid level. The other sensor 130 may be alternatively connected to the pump input 118 by the line shown as a dashed line 132, where it can determine the amount of flow of the fluid entering the input 118 and the percentage of the particles suspended in the liquid part of the fluid being removed from the wound 114. It is after this sensor's location that the separation of the air and the liquid fluid and the particles suspended in it takes place. The pump impeller therefore receives very little, if any, of such liquid and its suspended particles, and the pump outlet 120 discharges clean air into the atmosphere. While not shown, a filter may be provided in the pump outlet 120 to filter out any of the liquid and particles suspended in it that were not removed by the separator. A filter, not shown, may also be located between the separator, not shown, and the pump impeller, not shown, to trap any of the liquid and particles suspended in it that was not removed by the separator. The pump assembly may have a gauge 138 that has a pressure sensor, not shown, that also reads and indicates the differential subatmospheric pressure that is being created and may be connected to provide that information to the pump control 110 through computer 140 and the wire 133.

The input section 104 is usually a part of the computer 140, but is shown separately for schematic purposes. The other parts of the data and control system may also be integrated with the computer 140, but are also shown separately for schematic purposes. The computer 140 has a part 142 of it shown as a front view of the computer case 146 and another part 144 of it shown as a side view of the computer case 146 for schematic purposes. The source of electricity 106 is connected by wires 148 to the power-receiving input 150 of the computer. There is a space provided in the computer case 146 for a back-up battery 152. This back-up battery is preferably one that has sufficient electrical storage capacity to power the entire system for an extended time. The back-up battery, when it is fully charged, should last for at least about two hours when in use, and it is desired that it last even longer if it does not become too cumbersome. It is to be charged at any time that the power source fo6 is connected to the power-receiving input 150. The back-up battery 152 is connected to the motor of the pump assembly 108, through the pump control switch 198 as later described, so that it can also power the pump when there is no outside power available, but that connection is not shown. A part of the data and control system preferably includes a power switch that is activated by a loss of power to electrically connect the back-up battery to the system. Thus it can be similar to back-up power packs marketed to keep one's computer running even though the regular power source is disconnected for some reason. By having this independence, the system can be used wherever needed to be able to provide treatment to the wound 114 without the necessity of immediately being shut down for lack of power. When it is needed to carry the computer 140 and the rest of the system, a strap may be fastened to the two strap holders 208 and 210 secured to the computer case so that it may be safely transported in this manner. When the fluid storage container 112 is detachable, it should be removed before carrying the system using a strap. This is a desirable way to move the entire system from one patient's room to another patient's room when the entire system 100 except for the fluid storage container 112 is not readily detachable, and moreover is often made to all be contained in one case. It is not only easier to carry then, but it is less likely to be dropped and damaged. At times the entire system is placed or even built on a cart, and then the cart may be readily rolled to many parts of a building without having to do any more than unplug it from power socket in one location and to plug it into another power socket in the second location.

The side view 144 of the computer case 146 shows four receptacles 154, 156, 158 and 160. These receptacles have the wires 160, 162, 164 and 166, respectively, received in those receptacles. These wires also are shown as being respectively connected with sensors 124, 126, 128, and 130. As earlier noted, the wire 172 leading from the computer 140 via receptacles 154, 156, 158 and 160, may be alternately connected by wire 132 to the area of the pump intake 118 so that it can sense a condition and receive data concerning that condition, and then transport that data to the computer 140 through receptacle 160. Similarly, the wire 166 may be alternately connected by wire 133 to the pump control 110 via the computer 140.

The computer 140 is also connected with the Mode Select Panel 170 via wire 171. That panel has four buttons 172, 174, 176, and 178, or the equivalent, on a touch screen. These buttons are respectively identified to relate to the four modes in which the system may operate by their labels of No. 1, No. 2, No. 3, and No. 4. These four modes may have such respective names that are word reminders of the relative strengths of those modes, as “Aggressive” or “Very Strong,” “Relaxed” or “Strong,” “Soft” or “Medium,” and “Gentle” or “Light.” A set of such word reminders are usually provided on the buttons, with the button shown as having No. 1 on it being labeled as Aggressive or Very Strong, button No. 2 on it being labeled as Relaxed or Strong, button No. 3 on it being labeled as Soft or Medium, and the button No. 4 on it being labeled as Gentle or Light. The four buttons may be of the type that is pushed to turn it on, and pushed again to turn it off. Only one of the four buttons is to be pushed to turn it on, and electrically complete its connection across its wire to the Mode Select Panel. After one selection is connected to the Mode Select panel, and is entered by pushing the Enter button 179, that selection is sent to the computer 140, and no others are connected to the Mode Select Panel. If a different button is pushed and the Enter button is pushed, that button becomes active and the one that was active in deactivated. They may be arranged with lights that only one can be lighted, and that would be the one selected. If the mode is to be changed, and the Panel does not have an automatic change of energized buttons when a different one from the active one is pushed, the one that is lighted indicating that it is the one that is connected to the Mode Select Panel has to be pushed to turn it off. Then a different button may be pushed to select a different Mode. The Modes are selected by the patient's negative reaction to having a certain pressure such as that chosen to be the pressure to begin the treatment as the equivalent to the pressure that the No. 2 button represents. If the differential subatmospheric pressure selected is equal to the pressure represented by the No. 2 button, that button will be on. With the system turned on, with the wound dressing having been applied, the person controlling the treatment session will ask the patient to describe the amount of discomfort that he or she feels. It the reply is that it is comfortable, then the selection should be changed to the No. 1 button, the differential subatmospheric pressure associated with it being somewhat greater. It that feels only mildly uncomfortable, then that is the mode to be selected. If it is very uncomfortable, the selection should return to that differential subatmospheric pressure represented by button No. 2. If that selection is now still very uncomfortable, the selection should be changed to that differential subatmospheric pressure represented by button No. 3. If it is the one where there is only minor discomfort, it should be selected for the treatment. If it is still very uncomfortable, the selection must be changed to the differential subatmospheric pressure represented by button No. 4. It may be that in future treatment sessions, the patient can reasonably tolerate the higher pressure for the next higher level of differential subatmospheric pressure. It is usually preferable that the No. 1 selection be employed if at all possible, because this increases the range that can be modified when some sensor or sensors request that the differential subatmospheric pressure be increased. By way of example only, button No. 1 may set at an initial base differential subatmospheric pressure of 100 mm Hg, button No. 2 may set at one of 90 mm Hg, Button No. 3 may be set at one of 80 mm Hg, and button No. 4 may set at one of 70 mm Hg. If a different pressure is desired, it may be manually entered into the computer. The manual setting can be any integral number of mm Hg that the pump is allowed to produce. Because of noted problems with higher pressures at times, that maximum allowed number should be less than 125 mm Hg. Then, should different covers and dressings be used that are not up to the standards set herein for them, it still would be unlikely that the differential subatmospheric pressure that may increase the danger to the patient is not allowed to be set as the initial differential subatmospheric pressure to be used.

The four readout stations 180, 182, 184, and 186 are each connected to show the data that each of four sensors 124, 126, 128, and 130 are sending back to the computer. This is a way for the person or persons tending the patient during this treatment to see what is presently being received as to the condition of this measured information. It is also a check to see that the system is operating with each of the sensors being operational.

The two switches 196 and 198 are respectively labeled as being for the system and the pump 118. They are connected by appropriate wires They are shown as being in a neutral position, but being toggled upwardly toward “ON” and to be toggled downwardly from the neutral position toward “OFF” depending upon when the person in charge of this treatment is ready for the operation of the wound care system 100 and the operation of the vacuum pump 108 to begin. They are also used to turn the system and the pump off.

The keyboard 200 and the digital keypad 202 may be either integral or separate as appropriate. They are respectively connected to the computer 140 by wires 204 and 206. They are used for inputs that the computer screen will ask for when preparing the system for treatment of a specific patient.

The monitor screen 188 is connected to the computer by the wires 192. Like most computers, the monitor screen is separate, but it is also desirable to have it mounted as a part of the computer. This may be more feasible than for ordinary computers and monitors because this monitor screen may be somewhat smaller than the 19 to 25 inch monitor screens that are now available. It is also desirable, because the entire system can be integrated into one housing if desired, with the fluid storage container having a place for it while it is also easily removed and replaced.

The monitor screen 188 will have several points of information shown as may be desired. The person running this treatment will enter all of the information required for being entered into the system in order to use the system for a treatment session. It will also show any potential incorrect solutions or problems within the entire system 100 at all times that all have been sent by the computer. The system can be used in a manner that, when starting the treatment, it is already set up, and when starting it, all data coming in is not only shown, but is also delivered to the proper places in the system, and the output differential subatmospheric pressure is that set to be used. By way of example, when you are asked to enter the name of a patient, and there is already a record for that patient in the data file, the user can call up that data. This is also convenient because the data also can show the prior treatment sessions for that patient. Data for all patients and all treatment procedures are preferably kept in the computer, and also in a separate data storage facility or archive device. Connection point 212, located on the computer case side 144, is provided to attach a cable, which may be a USB cable, connected to an electronic archive device where all the data and information about each patient is archived. It is through this connection point that data on each patient may be recalled as needed and viewed on the monitor screen 188. Of course, it may also be recalled from the archive device, which may be a portable hard drive or a flash drive by way of example but not of limitation, on a separate computer when that is appropriate.

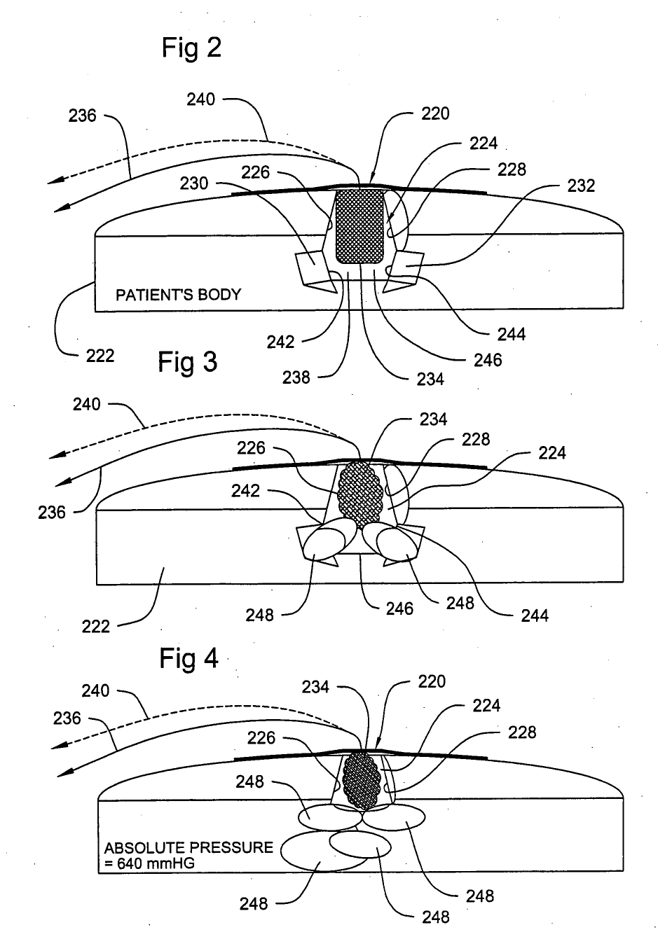

FIGS. 2, 3, and 4 shows what often happens as the process of the treatment proceeds when the currently standard insert 234 is used irrespective of the shape of the wound 224. Insert 234 has a rectangular cross-section and it is of the “one-size-fits-all” category is used. As shown, the insert 234 just does not fit the wound 224. The cover and dressing 220 is shown as being installed over the patient's body 222, and the dressing insert 234 has been placed in the wound 224. These elements are shown in cross-section to better illustrate the changes that take place. Wound 224 is shown in an all too typical shape. It has walls 226 and 228 extending downwardly and further apart as the depth increases. It also has some misshapen parts forming hollow spaces 230 and 232 in the opposite corners. It is also larger in cross-section than the insert 234 so that there is space between the wound walls after the insert 234 has been inserted. The insert 234 extends for much of the length of the wound, having been cut to its length from a long length of material from which several inserts of different lengths as needed may be cut.

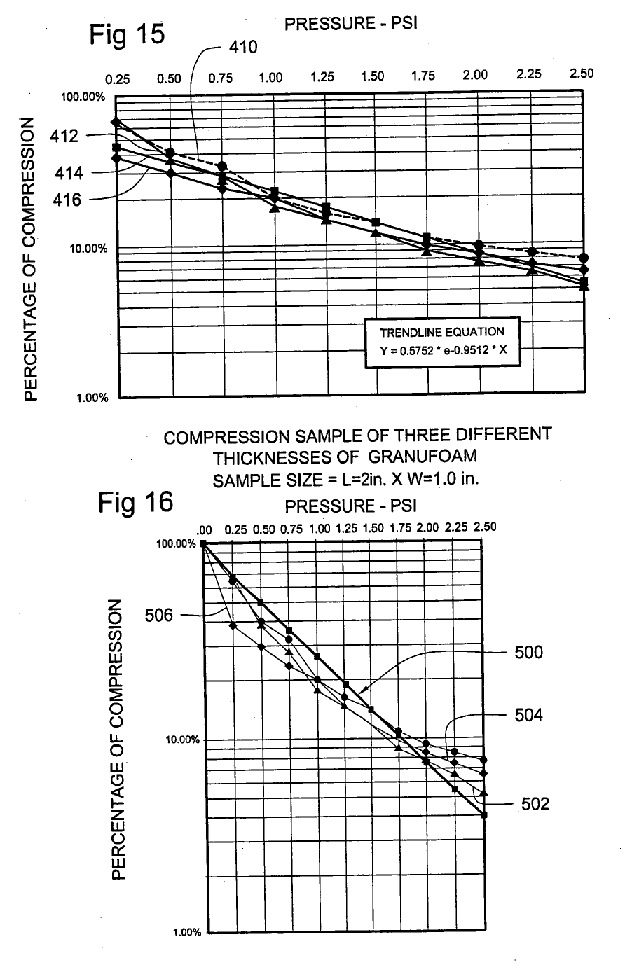

From information available, and samples bought and tested well before the provisional patent application on which this application is based was filed, characteristics of the dressing insert 234, such as tensile strength, compressibility, porosity, pore size distribution, permeability and structural integrity had apparently had not been adequately considered, because such inserts having the needed characteristics had not been introduced on the products. See Sample 1 of FIG. 15, showing how the production insert did not meet standards with regard to compressibility. The sequences progressively shown in FIGS. 2, 3, and 4 are assumed to be run with a control mechanism that has been in use for some years, and using the type of procedure that has become common with the major systems currently in use. As the wound being treated is relatively large, the person in charge of the procedure has set the differential subatmospheric pressure to a setting of 120 mm Hg at day 1, and the insert 234 was not changed for one of a different shape or having different properties during the treatment time. Assuming that the ambient atmospheric pressure was the standard 760 mm Hg, the absolute pressure in the wound while the treatment is in progress would be 640 mm Hg. This subatmospheric pressure is delivered from a vacuum pump by tube 236 to the wound cover and dressing 220 so that it is in the wound cavity 238 via the insert 234. The wires that make up a data cable 240 are also connected to sensors, not shown in these three figures. FIG. 2 shows the setup just before the treatment session is started. It is started, and the 120 mm Hg subatmospheric pressure evacuates the womb cavity 238 and the insert 234 begins to be compressed. While the wounds walls 226, 228, 242, 244, and 246 do not move in enough to engage the insert, because the insert is not properly shaped, there are hollow spaces left in parts of the wound, and in particularly in the wound's innermost lower corner spaces 230 and 232. This has the result of the development of the tunnels or fistulas 248, shown in FIG. 3, as the wound otherwise becomes smaller and smaller as seen in FIGS. 2 and 3, and in doing so, it may be compressed to the extent that it has little or no porosity left. All of these pressures, combined with the shape of the insert and its lack of sufficient resistance to compressibility, often have resulted in the development of the tunnels or fistulas 248. These tunnels or fistulas are often closed off from the part of the wound cavity in which the insert 234 is located when the rest of the wound seems to be healing, and they usually are infected, so that they become a serious problem requiring different attention and procedures. Documented treatments for that condition have taken as much as 77 additional days to treat those infected tunnels and have the wound heal properly. Such dangerous conditions have been found to too often result in a grossly retarded complete and safe healing, requiring weeks of corrective treatment with unnecessary pain and suffering, as well as cost.

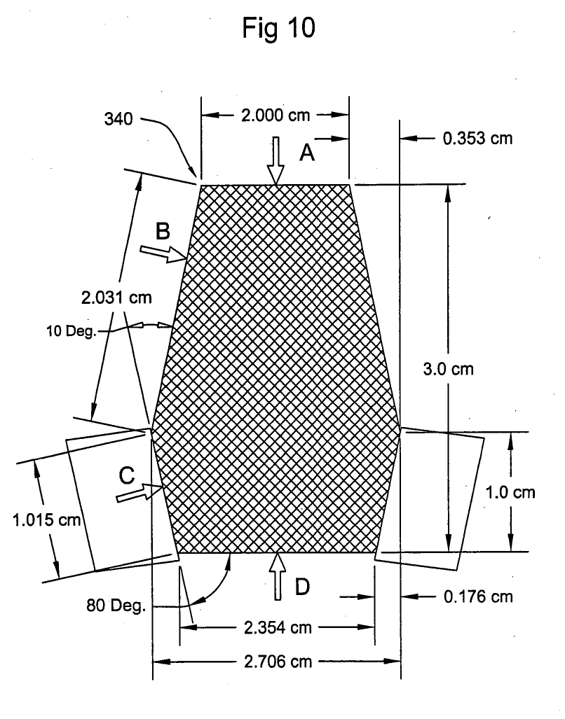

FIG. 5 shows the cover and dressing assembly 300 of the invention as it is before treatment has started and therefore the wound 302 is at ambient atmospheric pressure. The cover and dressing assembly is comprised of the following parts: A differential subatmospheric pressure blanket 304 with a lower permeable layer 306; a cover 308 that has a lower horizontal part 310 that engages the tissue 312 of the patient that surrounds the wound 302 and is shaped somewhat like an oval to the extent that the wound opening through the tissue 312 with the wound opening edges may be said to be somewhat shaped. The term “oval used here is not a word of limitation, but is a simplistic way to roughly describe the general appearance of the wound opening. Of course, at times it may appear to be almost a slit, sometimes it may be generally L-shaped, and at other times it may be rough and jagged so that there is not a term of shape that would describe it. Anyway, the wound opening has some sort of shape and the lower horizontal part 310 of the cover 308 covers that shape over the tissue 312 that surrounds the wound opening in the same general shape as the wound opening. The cover 308 also has a vertical part 314 which is similarly shaped to the inner side 316 of the lower horizontal part 3 10, and extends upwardly from that lower horizontal part 310. The cover then has an upper horizontal part 318 that extends substantially parallel to the lower horizontal part 3 10. The inner side of the upper horizontal part 318 forms an opening 320. The dressing blanket 304 is received inside the cover 308, and is shaped to have its outer surface band 324 to be engaged with the inner side of the upper horizontal part 318, and a part of the top 326 of the blanket 304 engages the under side 328 of the upper horizontal part 318. The dressing blanket 304 is comprised of two layers. The layer of the blanket which is porous and permeable must undergo the same tests of compressibility, porosity, tensile strength, pore size distribution, structural integrity, and permeability as the wound dressing material is to undergo, as detailed below, and the optimum properties may not necessarily be the same. The blanket is prepared with the assistance of a layer of 3M's Tegaderm™ or equivalent which is used to seal off the permeable lower layer of the blanket such that the differential subatmospheric pressure imposed will seal the blanket against the patient's body areas that surround the wound. The blanket will be used in all cases where a wound dressing implant is used, but may also be used alone when the wound is very shallow. The upper layer 330 is a permeable layer of material that has sensors 332 and 334 in it. The lower layer 336 is a material that meets the requirements of permeability, structural integrity, bio-reactivity reactions, and tensile strength set above. The bottom side 338 of the blanket 304 engages the patient's tissue 312 immediately surrounding the wound 302. The insert 340 is shaped to the shape of the wound and is received inside the wound. In this instance, it substantially fills the wound so that the wound sides are near to or in surface engagement with the insert sides. Refer to FIG. 10 for more details of the cross-section shape of the insert.

In this arrangement, the cover and dressing assembly 300 has a seal cap 342 which contains the data sensors that extend into the upper layer 330 of the blanket 304. The seal cap 342 provides a secure connection against leakage at the point where the tube 240 connects to permit the differential subatmospheric pressure produced by the pump 108 under the control of the data and control system 102. It also protects against leakage around the wires 240 for the sensors 124, 126, 128, and 130, shown in FIG. 1.

When the system is turned on, the computer system has been provided with the information required as set forth earlier, and the pump begins to evacuate the wound interior until it quickly reaches the set amount of differential subatmospheric pressure set into the system by the computer in accordance with the data received by the computer and the guidelines built into the computer. Once that set differential subatmospheric pressure is attained, the pump keeps the wound interior at that pressure, subject to modifications being made to it, as earlier described. The subatmospheric pressure is also present on the underside and throughout the thickness of the blanket, and one may say that it acts on the cover inner surface to seal against leaks of atmospheric air into the system where the cover engages the tissue of the patient and the top of the blanket upper layer 218. Actually it is the ambient atmospheric pressure that acts on the cover and dressing outer surfaces because of the lower pressure acting on the cover and dressing inner surfaces. This force exerted by the ambient atmospheric pressure forces the blanket 304, and particularly the blanket lower layer 306, also in sealing relationship with tissue 312 of the patient and also forcing the cover 308 into sealing relationship with the outer surface of the blanket. At the same time it is acting on the vertical part 314 of the cover to move a part of that vertical part 314 into engagement with all of the outer band surface 324 of the blanket 304, and to also move the horizontal lower part 310 inwardly and to be urged by that same ambient atmospheric pressure into sealing engagement with the tissue 312. As shown in FIG. 6, the lower layer of the blanket 304 is compressed substantially by the pressure acting on the layer that also covers the open top of the wound 302 and the top of the insert 340. The differential subatmospheric pressure in the wound will allow the higher substantially ambient atmospheric pressure that exists in the patient's body to put some pressure on the insert. Due to the shape of the insert, shown in cross-section, it is being urged to stay within the wound. Also the wound walls are being urged into good engagement with the insert outer surfaces. This engagement of the insert and the wound walls, including the bottom wall, if any, results in no tunnels or fistulas being created in the lower interior part of the wound. When the wound is healed, it is cleanly healed.

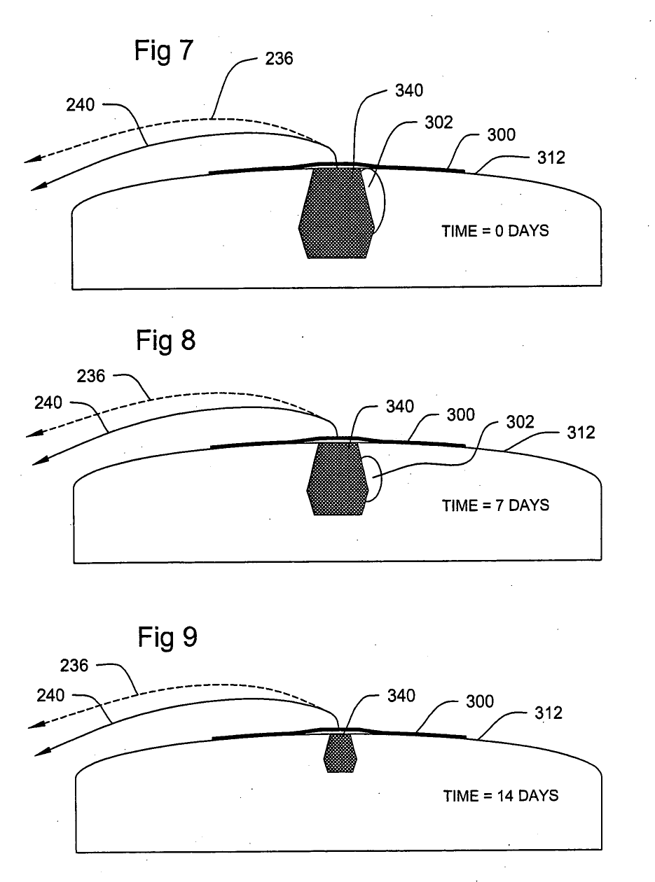

FIGS. 7, 8, and 9 are similar to FIGS. 5 and 6 insofar as the insert member 340 and the wound 302 are concerned. Other parts of the cover and dressing shown in FIGS. 5 and 6 have been omitted for simplicity. These FIGURES show the progression in healing, starting at the instigation of the treatments, and continuing for about 14 days, when the healing process has approached the time point where it is well on the way to healing without problems. In FIG. 7, the insert 340 has been placed within the wound 302, the cover and dressing are in place, all connections to the computer and the pump have been made, and all data have been entered. In this arrangement, a typical differential subatmospheric pressure of 90 mm Hg may have been selected, and the Aggressive or Very Strong mode selected. After there have been seven days of treatment, the condition is shown in FIG. 8. There may have been some incremental changes to the differential subatmospheric pressure delivered to the wound as one or more of the sensed conditions may have been sufficient to require a change which the computer requires the pump control to make. After this week, the delivered differential subatmospheric may have been decreased to 84 mm Hg, or increased to 93 mm Hg, for example. This is as it should be. It can be seen that the wound 302 and the insert 340 are both somewhat smaller. The wound has been healing from its bottom part, and the wound walls are still engaging the side and bottom surfaces of the insert. It is noted that the insert 340 has been compressed to that somewhat smaller size. Therefore, the properties that it has, such as compressibility, porosity, permeability, pore size distribution and structural integrity have served it well. After another seven days of treatment, the condition is shown in FIG. 9. The wound 304 has healed a great deal more, so that it has been decreased in size to about ¼ of its original cross-section area, and the insert 340 has also been decreased in size about the same amount. There are still no tunnels or fistulas forming, and the healing process is well on its way to a successful completion, probably within the next week or two.

FIG. 10 shows a cross-section of the insert 340 shown in FIGS. 4 through 9. This FIGURE is provided to provide the analysis of forces acting on the and the insert. It has six sides. They are the top side A, the upper left and right sides B, the lower left and right sides C, and the bottom side D. The size of the wound is expressed in centimeters, as normally measured by the medical personnel, and the forces and pressures are expressed in pounds of force and pounds per square inch (p.s.i.) of pressure. The forces are calculated as being directed vertically and horizontally, but, in reality, the forces have vectors acting perpendicular to the variously shaped sides of the insert 340 and the walls of the wound, so those forces have also been calculated as acting on the sides of the insert and the walls of the wound. Forces on the sample insert are exerted by the normal pressure in the patient's body, which is at the ambient atmospheric pressure and occurs because the pressure within the wound is at the lower differential subatmospheric pressure. This figure also shows how important it is to have the properly shaped wound dressing to always support the wound walls as differential pressure is applied. The figure uses an example differential pressure of 75 mm Hg as the differential subatmospheric pressure which is equal to 1.45 pounds per square inch (p.s.i.) acting on the sides and walls noted. The example wound dressing insert 340 is shaped to fit the wound with side walls sloping outwards and downward at an angle of approximately 10 degrees, and then a shorter reverse slope approximately 80 degrees. The calculations are provided below to explain how this shape allows the incident forces on the side walls of the wound to result in both lateral and vertical forces. It is the net force vectors of the forces which hold the wound dressing in its proper place against the wound walls as the differential pressures are induced. The wound dimensions, the pressures, the forces, the wound dressing dimensions, and the differential pressure setting, etc. are all illustrative, and will change with each patient treated, and with each dressing change—the main reason that a “one-size-fits-all” approach is inviting disaster, and mode selection considering wound size and patient tolerance is a much more viable approach. There are six surfaces on the exterior of the insert 340. There are pressures from the differential subatmospheric pressure being provided in the wound. In order to have measurements of pounds of force being exerted, there is a recital of the p.s.i. that will be acting on those surfaces. Calculations show that there is a net force of 0.470 pounds of force acting to hold the insert in place. The ratio of the areas of C/B have been calculated to continue to have a positive force acting to hold the insert in place, and that preferred ratio is about 0.5. However, that ratio has a range from about 0.05 to 0.85 where the net forces tend to hold the insert in place in the wound. That ratio is important, because one of the problems with inserts in the wound of this general type that are square or rectangular is that they do not have any vectors of side forces that tend to hold the insert in place, and it is more likely to move away from it should they remain in the wound. While some typical measurements are shown, they are only used to show some mathematical computations relating to the improved performance of this type of insert as compared to the rectangularly shaped, in cross-section, of the insert that has been used for some years. The important features are its shape and its contribution to the net forces tending to compress the insert while urging more upward flow of air and fluid entering the insert and moving upwardly, particularly nearer the bottom of the wound. This additional upward flow in that area seems to have the effect of being less likely to have tubes or fistulas form, as well as decreasing the differential atmospheric pressure usually used so that the insert is not so compressed that it can allow little or no flow through it.

There is no net upward flow of the air and the fluid as it enters the insert from either side of a rectangular insert. It starts out as being perpendicular to the side surfaces. The length of the insert does not come into play because it has no effect on the ratios of the opposed forces created while any pressure is action on all of the insert's outer surfaces once the widths of the surfaces are established. By widths, the measurements of each surface, as shown in FIG. 10, are the widths of the respective surfaces. When using the rectangular insert that has been used for some years, given the fact that its width is the same for all of its surfaces, there is no net vertical force vector acting on the insert vertically attributed to the side surfaces. Since in the installation of inserts have their upper surfaces being exposed to the full differential subatmospheric pressure discounts the downwardly acting force that would theoretically be there, because the upper surface of the insert is in engagement with the dressing where the differential subatmospheric pressure is applied, and in that area the air and fluid that enters the sides and bottom of the insert are drawn out of the insert and into the tube supplying the differential subatmospheric pressure.

When considering the insert such as that shown in cross-section in FIG. 10, for a mathematical analysis of the action of these forces, simple length for each of the surfaces such as 10 cm can be assumed. Under this assumed condition, the area of surface A is 20 cm, the area of surface D is 23.54 sq.cm, the areas of each surface B, B is 20.31 sq.cm, and the areas of each surface C,C is 10.15 sq.cm. Assuming that there is a differential subatmospheric pressure of 60 mm Hg, the absolute pressure acting on the insert 340 at the standard atmospheric condition is 700 mm Hg, or 7.00 cm Hg. That pressure then creates the respective values of the forces as follows: Force A would be 20 sq.cm×7.00, or 140.00 cm Hg if it were not the exit area for air and fluid within the wound that has entered the insert through its sides and bottom. Therefore, the supply of the differential atmospheric pressure at this point is exerting an upward force on the insert. Force D is therefore 23.54 sq.cm×7 cm Hg, or 164.78 cm Hg. Therefore the net vertical force considering only areas A and D would not be 24.78 cm Hg, acting upwardly. Each Force B is 20.31 sq.cm×7 cm Hg, or 142.17 cm Hg. Each Force C is 10.15 sq.cm×7 cm Hg, or 71.05 cm Hg. The net vertical force of force B as well as force C is the force acting on the actual area less the force that would be acting area calculated using vertical height of area B as well as that of force C. The vertical force acting on area B would be 2 cm×10 cm (20 cm)×7 cm Hg, or 140 cm Hg. Subtracting that from the actual force created on each area B, which is 142.14 cm, the vertical force acting on area B is 2.14 cm Hg, acting downwardly. The net vertical force acting on area C, calculated in the same manner as that for area B, that net vertical force is 71.05 cm Hg−70.0 cm Hg, or 1.05 cm Hg acting upwardly. Therefore, the net vertical force acting on both areas B and C is 2.14 cm Hg−1.05 cm Hg, or 1.09 cm Hg acting downwardly. With this configuration, the net vertical force acting on the insert 340 would 24.78 cm Hg−1.09 cm Hg, which is 23.69 cm Hg, acting upwardly, but we have to change the downward force A to an upward force, because it is causing the air and fluid to be moved out of the insert to the supply tube supplying the differential atmospheric pressure.

It is desirable to have some net upward forces acting particularly on the sides of insert, forcing the air and the fluid within the wound and surrounding the insert to also move upwardly as well as inwardly of the insert, passing through the porous insert via surface A and back toward the vacuum pump, thus increasing the probability that the material that is in the fluid within the wound will be more likely to be removed from the wound, and decreasing the possibility of the formation of the tunnels or fistulas. Using the general shape of an insert like that shown in FIG. 10, the upward flow can be increased as compared to the use of a rectangular insert.

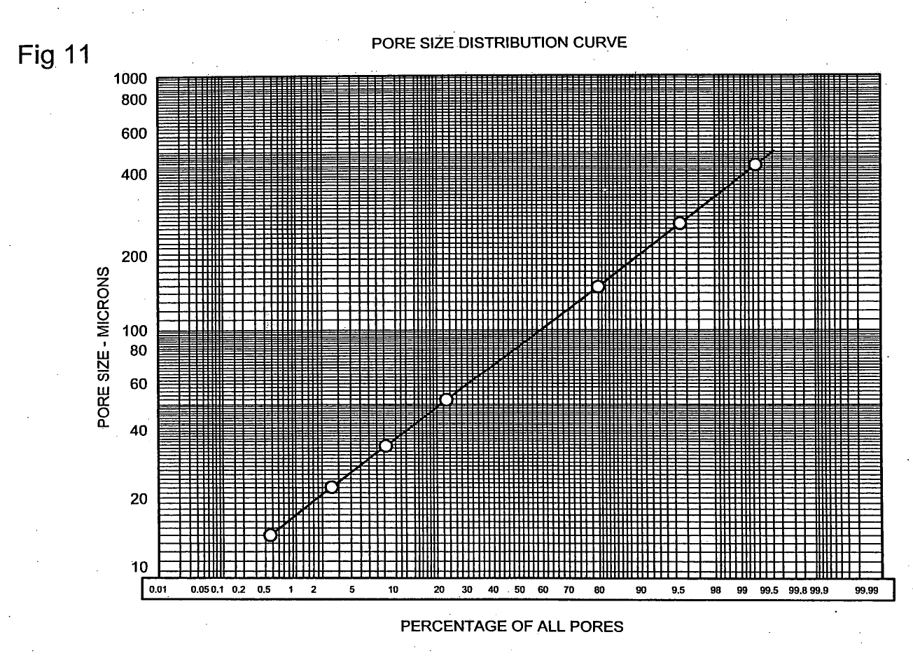

FIG. 11 is a graph which shows, as an example, the typical result of the type of which must be run to select the optimum wound dressing material considering the property of Pore Size Distribution. The values in the graph show an example of a dressing material for the insert 340 of FIG. 10 that has a higher percentage of pores of a certain that is the requirement for a satisfactory percentage. Yet, they are example figures based on only one porosity test for one particular material. This property of wound dressing material is important to enable the selection of a material which will preclude, to the greatest possible extent, the incursion of healing body cells into the wound dressing during the differential pressure therapy. All natural and man-made porous materials exhibit this kind of Pore Size Distribution, i.e., a straight line correlation on a log—probability graph. The correlation is extremely important in determining if the proposed wound dressing material has a preponderance of pores of a size such that healing cells in the human body will, or will not, invade the porous matrix. It is desirable that over half of the pores have a pore size of no more than 100 microns in order for the material in the insert such as insert 340 of FIG. 10. The sample figures this graph show that the sample tested had 50% of its pores with a size of about 80 microns or less, and had 60% of its pores with a pore size of 100 microns or less. Therefore the material that was tested and yielded this result was more than just satisfactory. It is quite desirable. This property will also have a large effect on the permeability value of the material. Yet, materials of the same porosity can have vastly different permeabilities due to other reasons than porosity.

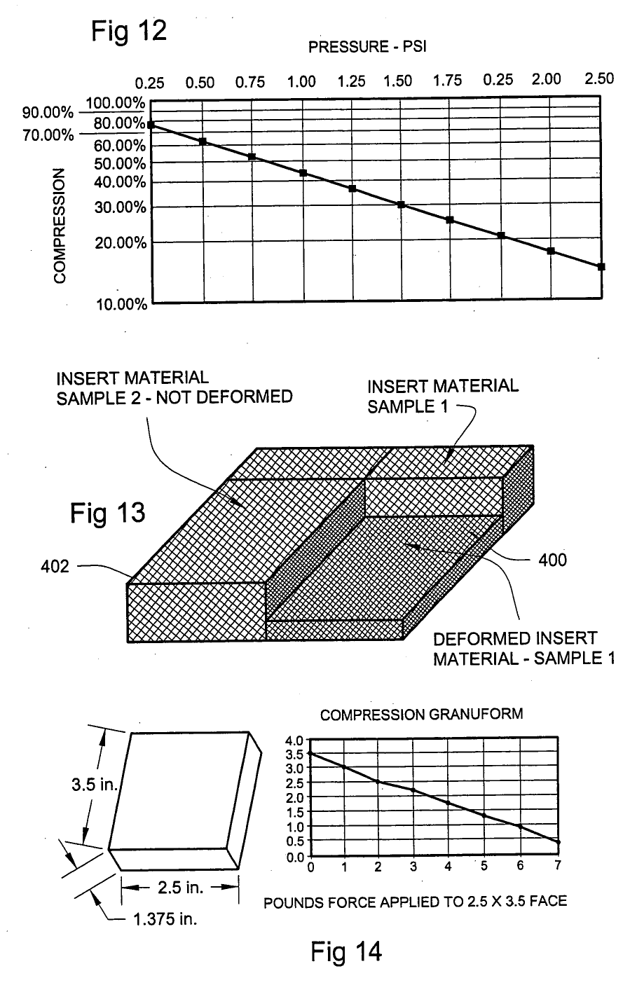

FIG. 12 illustrates the percentage of compression in relation to the pressure applied to a sample of material (sample 2 in FIG. 13) that has been considered for use as a dressing insert. This presentation shows a data sheet which will be used to collect data on samples which have indicated acceptable “compressibility” properties during the screening tests. There are several additional tests that each sample must pass prior to being accepted as a candidate for use in the CAPE™ mode of wound healing. The data developed with this graphic comprise some of the basic data for the computer program to be the controlling mechanism of the CAPE™ mode of wound healing. The system preferably requires a wound dressing, including the insert, to have very specific properties of which, along with variables selected by the medical operator, will exactly control the parameters of the healing modality to be imposed on the wound. While other wound dressings will undoubtedly be used by some using the system, they are not expected to obtain as good results as would be attained using a dressing that is optimal for the system unless they meet the stringent requirements set forth for a CAPE™ dressing concerning compressibility, permeability and porosity, among others mentioned herein. Some materials that may be considered eligible to serve as acceptable dressing material have different percentages of compression when a range of certain pressure values likely to be encountered by a dressing insert is applied to it. Such tests have been made on two very different samples 400 and 402 that can be considered to be an acceptable material for the dressing insert 234 shown in FIGS. 3 and 4, or the dressing insert 340 shown in FIGS. 5, 6, 7, 8, 9, and 10. The material of which at least most of the materials used in production for at least the past several years is the material that was for the dressing insert 234, and was the fresh insert material 400 of sample 1, shown in FIG. 13. The other insert material 402, which is sample 2 as shown in FIGS. 13 and 14, is a material that passed the compression test without compression-caused deformity. The material, not yet being used in production, of which a dressing insert should be made and should have a high degree of compression recovery after having been compressed at pressures likely to be encountered in wound therapy use. The recovery from compression testing, with the tests being identical as to the samples 400 and 402 having the same dimensions, and having the same face size. Two different typical test-sized materials were used. The compressibility of a “Granuform” sample 402, sample 1, is plotted in FIG. 14. The side-by-side comparison of the two materials is shown in FIG. 13, with the material 400 of which the insert 340 has been made, and the material of sample 2, which is 402. After the compression tests were completed, the sample 2 rebounded to substantially its same thickness, while the sample 1, identified as sample material 400, was deformed to about ⅓ of its thickness, as clearly shown in FIG. 13, remained at that deformed state for a long period of time, and never fully recovered as did sample 2. The undeformed part of sample 400, which is sample 1, is shown placed at one end of the tested sample so that a visual comparison is readily made to the before-and-after showings. The insert 340 mentioned earlier was made of the same material with the same characteristics as the sample 2 of FIG. 13. As shown in FIG. 13, it had quickly recovered from the compressibility test so that it returned to substantially its same thickness.

FIG. 14 speaks very well for itself, and no other comment is considered necessary.

FIG. 15 shows a graphic depiction of the results of a laboratory device that measures the compressibility of a sample of wound dressing material, and its application to the test of a type of wound dressing presently in wide usage. The accuracy of the device is ±0.005 pounds force and ±0.25 cm. It is used primarily as a screening device to eliminate materials which are greatly outside of the acceptable norms of compressibility. Tests were made upon three samples—all 2 in.×1 in. in area, and×3.4 cm, 2.5 cm, and 1.7 cm in thickness. The results showed that all the samples produced results which could be fairly accurately predicted for this material by the trend line 410 shown using a line that has small squares on it at each pressure noted for the vertical graph lines. That line on the graph is in accordance with the formula Y=0.5752*eˆ(−0.9512*X). The graph shows three different thicknesses of the same material having been tested. All three thicknesses, 1.7 cm, 2.5 cm and 3.4 cm, were tested, and all of them were very near to the trend line 410. Therefore, they would be acceptable insofar as this test was concerned. The graphic lines for the 1.7 cm thickness of material is identified by the reference number 412, the line for the 2.5 cm material is identified by the reference number 414, and the line for the 3.4 cm one is identified by the reference number 416. Comparison of the trend line to the tests run on a particular material, not shown on the graph, showed that it would be a very poor choice for a wound dressing in pressure-assisted healing, as it was compressed to 20% of its initial volume at a differential pressure of only 58 mm Hg (1.12 p.s.i.). As the graph shows, the trend line 410 shows a compression of about 40% at that pressure. That material almost certainly would have lost essentially all of its porosity and permeability well before it would have been exposed to differential subatmospheric pressures between 75 and 125 mm Hg., and could only have severely inhibited the healing process. The default differential subatmospheric pressure used when setting up the system utilizing these wound dressings was 125 mm Hg, which is has been used as the standard by many of the systems in use for some years. That setting is more than twice the collapse value shown by the test. This device is the screening device with which to select samples of wound dressing material to denote a specific wound dressing designed specifically to be used by the inventive system of differential subatmospheric pressure-assisted wound healing.

In FIG. 16, there is a trendline for all three thicknesses is shown by line 500. The actual compression line for the sample that was 1.7 cm thick is shown by line 502. The actual compression line for the sample that was 2.5 cm thick is shown by line 504, and the actual compression line for the sample that was 3.4 cm thick is shown by line 506. The trendline 500 shows that the average pressure at which all three samples were compressed to 10% occurred with a pressure of 1.88 p.s.i. The one of the three samples that had the highest pressure needed to compress it to 10% was the 3.4 cm thick sample. The one of the three samples with the lowest pressure needed to compress it to 10% was the 1.7 cm thick sample.

FIG. 17 shows the magnitude of the subsequent shape and distortion due to the use of the high subatmospheric pressure of 125 mm Hg. The distortion shown is also the result of the resistence of the body parts to this distortion.

In FIG. 18, a pressure sensor senses the vacuum pump output, shown on the part 650 of the graphed pressure line on which the pressure in the wound is plotted, and when it is pumping shuts off the pump when the range upper level of 665 mm Hg pressure is attained, indicated by upper dashed line 665, and remains off so that the pressure at the wound decreases, as shown on the part 655 of the graphed pressure line, until the range lower level of 635 mm Hg in the wound is attained, indicated by the lower dashed line 635, at which time it turns the vacuum pump on. While the major producer of the VAC system calls this a “Continuous Mode” it is in fact a Cyclic Mode where the vacuum pump, when running, it is supplying a numerically larger subatmospheric pressure than the high end of the range set forth, and is not supplying any subatmospheric pressure so long as the higher absolute pressure at the end of the range is not reached as the pressure within the wound changes because of the leakage of ambient atmospheric air into the wound area, with the actual subatmospheric pressure being somewhere within the specified range, understood to have been about 650 mm Hg. Therefore, it is a cyclic Start-Stop-Start-Stop mode of operation. When it is in the Stop part of its cycle, the actual subatmospheric pressure slowly changes because there is no complete and perfect seal of the entire system, including the wound itself, and some ambient atmosphere air will (usually) leak slowly into that subatmospheric pressure area where the wound is located.

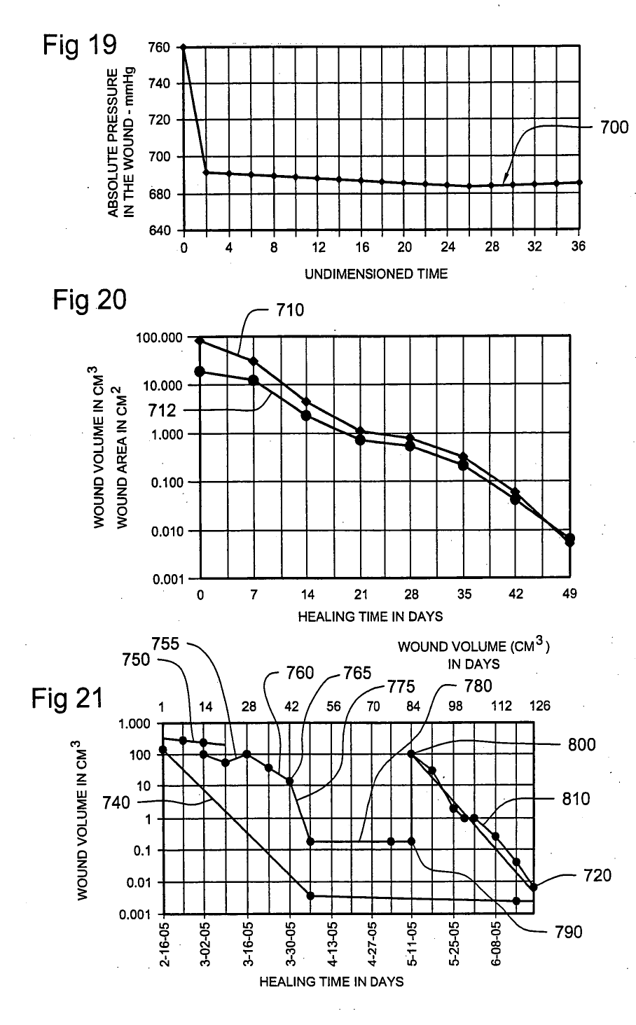

In FIG. 19, the differential subatmospheric pressure at any particular time is the difference between 760 mm Hg (assuming that the ambient atmospheric pressure at the time is the standard atmospheric pressure at sea level of 760 mm Hg. The points on the graphed line 700 are graphed on the scale of absolute pressure. The graphed values, shown as graphed points on the graphed lines, will always change when any of the modes of therapy are selected after having been set on a different therapy mode. The graphed points on the graphed lines can also purposely changed when the change in one or more of the sensed conditions results in the computer sending a signal to the pump control to increase or decrease the differential subatmospheric pressure delivered to the wound. Additionally, the time scale is left purposely without dimensions, as the scale will change, not only with the mode selected, but from day to day as healing progresses. The ability of this instantaneously changing data to be collected for later study will assist the health officials not only in designing the present treatment, but for the design of future treatments as well. In this graph, the system was started, and by time 2 had reached its selected differential subatmospheric pressure of about 692 mm Hg that had been initially set by computer. Over an extended time to time 25, the originally selected differential subatmospheric pressure has been slowly modified by the computer in very small decreasing differential subatmospheric pressure changes until time 26, and at the next undimensioned time 28, and thereafter through time 36, the computer had been gradually increasing the differential pressure by changes made from time to time. The difference between the cyclic manner of maintaining the desired differential subatmospheric pressure, shown in FIG. 18, and the manner of doing so as shown in FIG. 19, using the invention herein disclosed and claimed, is quite clear.

In FIG. 20, the plotted line 710 is the plot considering the volume of the wound in cm³, and the plotted line 712 is the plot considering the area of the wound in cm² It can be plainly seen that it is the logarithmic values plotted in FIG. 20 which show an almost linear relationship to time in normal healing. This is important as it shows that almost 50% of the healing takes place in the first week, and approximately 98% has taken place in the first 3 weeks. After this time, it is probable that differential pressure-assisted healing treatment will no longer be necessary.

FIG. 21 is a bit more personal, because it charts the actual experience of one of the inventors, during the time he was being treated with the standard 125 mm Hg that was so prevalent as late as 2005, about one year before the provisional application on which this application is based was filed. At that time there was no recognized need for a change in the treatment insofar as the makers of the treatment with which he was treated was concerned.

1. A wound care system using differential subatmospheric pressure applied to the wound to improve healing, said system comprising:
a source of differential subatmospheric pressure arranged to be applied to a wound, said source having a control for modifying the differential subatmospheric pressure to be applied, and also while being applied, to the wound;
a computer having a program for controlling said source control;
at least one sensor sensing a wound condition and any changes in that condition, said sensor sending data reflecting the wound condition and also reflecting any changes in that condition to said computer program of said computer, said computer program using said data from said at least one sensor and calculating what change, if any, should be made in said differential subatmospheric pressure being applied to the wound from said source of differential subatmospheric pressure, and sending a signal to said source control to make that change in said differential subatmospheric pressure, said source controlling said source of different subatmospheric pressure so that said change in said differential subatmospheric pressure is made.

2. The system of claim 1, said at least one sensor sensing the wound condition of the temperature within the wound.

3. The system of claim 1, said at least one sensor sensing the wound condition of the differential subatmospheric pressure within the wound and said data sent by said sensor to said computer program relating to any changes to said differential subatmospheric program being compared to said differential subatmospheric pressure to the last pressure change and determination if there is such a change that has not been authorized, and if so, then sending a signal for a technician overseeing the operation of the signal that an unauthorized-change has occurred.

4. The system of claim 1, in which there are a plurality of said sensors with each of said sensors sensing a different wound-related condition and sending data reflecting the conditioned being sensed by each of said sensors to said computer program, said computer program comparing any changes in said data sensing any changes to any of said sensed conditions and when there are at least two changes sensed, calculating the amount of changes to said differential atmospheric pressure in said wound and arithmetically adding said potential changes, whether either recommending an increase or a decrease in said differential atmospheric pressure in said wound, and sending a signal to said control controlling said source of said differential atmospheric pressure being delivered to the wound to change the that differential atmospheric pressure being delivered to the wound to the extent that the net change, if any, is changed.

5. The system of claim 1, said computer program also recording all data received and all changes recommended.

6. The system of claim 1, said computer program recording the record all data concerning each patient's treatment sessions and having means to recall said data when interrogated.

7. Said source of differential subatmospheric pressure being a pump that has its input operatively connected to said wound and its output pressure being discharged to the ambient atmosphere.

8. In a wound care system for wounds comprising:
a wound cover for creating a closed cover of a wound to be treated;
a pump assembly operable to produce subatmospheric pressures relative to the ambient atmospheric pressure within a pressure range useful in treating open wounds, said pump assembly comprising:
an air impeller and a motor connected to drive said impeller, said air impeller moving air in one direction with air pressure that is greater than the ambient air pressure and removing air from an enclosed space located under said wound cover and in doing so creating a subatmospheric pressure;
an pump air intake comprising a conduit connected to said wound cover to receive airflow from under said wound cover when said wound cover covers a wound to be treated;
a pump air discharge port through which from under said wound cover is discharged from said pump;
a control for said air pump assembly connected therewith to cause said motor to be turned on and off, and also to set the pressure value of the created subatmospheric pressure; the improvement comprising:
sensing means for creating data signals in accordance with information received by said sensing means;
a control and recording system by which information concerning a particular patient to have a wound treated by said system is received and recorded as well as receiving and recording said data signals from said sensing means;
said control mechanism being programmed to set a defined subatmospheric air pressure that said pump is to create in said pump air intake and under said sealed cover, said defined differential subatmospheric pressure being the differential between said ambient pressure and said subatmospheric air pressure and therefore remaining constant even when the ambient atmospheric pressure changes;
said control system creating modifying signals in accordance with said received data signals when said data signals provide information indicating the desirability of changing said defined differential subatmospheric pressure;
said modifying signals being then sent to said pump control system to modify the subatmospheric pressure in said pump intake conduit and in the enclosed space under said wound cover in accordance with the need to change said subatmospheric pressure and in so doing changing as least one of said sensed conditions relating to said wound and maintaining the differential subatmospheric pressure within the enclosed space under said cover at a changed value that improves the overall action of the differential subatmospheric pressure on the wound.

9. The system of claim 8 wherein said data signals from said sensing means in which there is a plurality of sensing means sensing different characteristics of information, at least one of which with a change in said differential subatmospheric pressure would increase the differential between the ambient atmospheric pressure and the current defined subatmospheric pressure to a first given extent, and another one of said sensed information which with a change in said subatmospheric pressure would decrease the differential between the ambient atmospheric pressure and the current defined subatmospheric pressure to a second given extent, with the result that all of the changes desired in a given increased or decreased extent are mathematically applied and the net result thereof is the subject of a signal sent from said control system to make the net change, if any is then still required, in the value of the differential between the ambient atmospheric pressure and a new defined subatmospheric pressure.

10. The system of claim 9 in which said information data signals representing different sensing means are weighted in accordance with the relative values of the potential changes based on different information sources relative values to the need for changing said differential between the ambient atmospheric pressure and the currently-defined subatmospheric pressure, so that a similar change amount in one set of information in relation to another change amount is said other set of information for the more heavily weighted information will be larger than the set of information of the lesser weighted set of information and will therefore have more influence on the final change, if any, sent to said control for said air pump assembly.

11. A system for treating a patient's wound using a differential subatmospheric pressure delivered to said wound, said system comprising:
a pressure source for establishing and maintaining a differential subatmospheric pressure that is the difference between the ambient atmospheric pressure and a subatmospheric pressure selected to be imposed on said wound from said pressure source for subatmospheric pressure treatment of said wound;
a first control mechanism for directly controlling the subatmospheric being delivered to said wound, said control mechanism being set to establish a desired initial differential subatmospheric pressure to be delivered to said wound to be or being treated, and being capable of changing that established desired differential subatmospheric pressure by a specified amount upon receipt of any signal to do so;
a second control mechanism comprising:
a plurality of sensing devices positioned to separately sense at least two conditions relating to a wound to be treated by said system and at least one of said plurality of devices being positioned to sense the current ambient atmospheric pressure, said at least two devices generating separate signals, each of which corresponding measurements of each of said at least two wound-related conditions that are sensed, and said at least one of said plurality of devices generating a separate signal that corresponds to the ambient atmospheric pressure;
data input and data receiving devices for the input and reception of data relating to a particular patient about to be treated, including name, address and other typical data required to be able to identify each patient that has been treated by said system and recall such data upon demand;
said data input and data receiving devices further including:
a data reception device receiving said separate signals; and
a data recording device recording said separate signals and the times said each of said recorded signals were received;
said data and data receiving devices also being arranged to receive and record new data as it becomes available relating to the particular patient while that patient is being treated by said system;
said control system still further comprising:
data input ports for inputting data that comprises (a) the patient's sensitivity to the subatmospheric treatment levels used to treat the wound of the patient; (b) an initially differential subatmospheric pressure to be established and delivered to the wound to be treated once the pressure source is turned on to provide a subatmospheric pressure to the wound to be treated; and other desired information that may be appropriate to the functions of said control system;
said control system processing said data received from said sensing devices and calculating any changes that should be involved in changing said established differential subatmospheric pressure when such data represents any changes in the condition sensed, such changes being weighted in accordance with the value to the wound treatment that each sensed conditions has, recognizing that one or more of such conditions may be of more or less importance in the changing of the differential subatmospheric pressure being supplied to the wound, and to calculate the result and desired change in said differential subatmospheric pressure being currently delivered to the wound; the result of said result and desired change being delivered to said pressure source first control mechanism which then changes the established differential subatmospheric pressure being delivered to the wound so that the changed differential subatmospheric pressure becomes the established differential subatmospheric pressure then being delivered to the wound, which is further changed from time to time in the same manner when said control mechanism shows that such change is appropriate;
Said sensing, receiving and recording of data and said calculations being a continous process so long as a differential subatmospheric pressure is being delivered to the wound being treated.
